(19) United States
(12) Patent Application Publication
(10) Pub. No.: US 2023/0405321 A1
(43) Pub. Date: Dec. 21, 2023

(54) METHODS AND SYSTEMS FOR AUGMENTING AND/OR SIMULATING FLAVORS

(71) Applicant: University of Maine System Board of Trustees, Orono, ME (US)

(72) Inventors: Ranasinghe Arachchilage Nimesha Ranasinghe, Orono, ME (US); Michael Gecawicz, Orono, ME (US); Jonathan Roman Bland, Orono, ME (US); Meetha Nesam James Ravindran Santhakumar, Orono, ME (US)

(21) Appl. No.: 18/013,056

(22) PCT Filed: Jul. 23, 2021
(86) PCT No.: PCT/US2021/042887

§ 371 Date: Dec. 27, 2022

Related U.S. Application Data
(60) Provisional application No. 63/056,202, filed on Jul. 24, 2020.

(51) Int. Cl. A61N 1/36; A61N 1/05

(52) U.S. Cl. CPC A61N 1/36034; A61N 1/0548; A61N 1/0546

(57) ABSTRACT

Presented herein are systems, methods, and techniques related to delivery of simulated flavor sensations to a user while consuming (e.g., eating, drinking) food items. The perception of flavor is a multisensory experience as it involves not only taste, but other sensory experiences such as sight, sound, smell, and touch. Delivery of one or more sensory experiences to a user as the user is consuming a food item is able to augment how the user experiences the food item's flavor. For example, changing a smell or color a food item has affects how users perceive it when being consumed Changing perceptions through stimulating various senses can alter expectations significantly and in unexpected ways when delivering or combining stimuli. Accordingly in some embodiments, the present disclosure provides for technologies (e.g., device(s), systems and/or method(s)) for delivering simulated flavor sensations while consuming food items (e.g., solid food items, a beverage).

### CROSS-REFERENCE TO RELATED APPLICATIONS

This application claims priority to and benefit of U.S. Provisional Application No. 63/056,202 filed Jun. 24, 2020, the disclosure of which is incorporated by reference herein in its entirety.

### BACKGROUND OF THE INVENTION

Flavors are the sensory impressions of foods and beverages that we consume every day and a vital component in human survival. The perception of flavor is a multisensory experience that involves information from many human senses (e.g., sight, hearing, taste, smell, and touch). Even though the prior experiences and expectations are involved in flavor perception, the perception of flavor is commonly narrowed down to the intrinsic flavor components, which are the sensations of taste and smell.

The taste of a food item is an intensely personal experience. It only takes a brief trip to a grocery store to see the vast array of items having different flavors. For example, food products such as sports drinks and coffees are available in a wide variety of flavorings as individuals vary significantly in their flavor preferences. The reasons these flavor preferences exist are many-fold and may be due to, among other things, region of origin, previous experiences with a flavor, age of the individual, or genetics.

In addition, condiments and items available not only for making food, but for changing the flavor of foods are quite plentiful and varied. Currently, if an individual wants to change the taste of a food item, such as coffee, there are a number of condiments and/or flavorings available. Sugars, milks, and creams are widely available and commonly used to change the flavor of coffee to suit an individual's desire.

However, as evidenced by the variety of condiments and flavors of foods available, it is difficult to craft flavors that appeal to a universal audience due to the individualized experiences surrounding taste, texture, or other attributes of a food or beverage. Furthermore, the addition of many of these condiments can also create undesirably inconsistent experiences from one bite or sip to the next. Additionally, many of the additives and condiments that make food flavorful and enticing are also unhealthy. Customers have few options for low-calorie and healthy alternatives to many of these options. Moreover, many of the low-calorie options are unsatisfying and may present health problems themselves.

Therefore, there is a need for improved technologies for the delivery of individualized and customizable tastes and/or tasting experiences to individuals.

### SUMMARY

Presented herein are systems, methods, and techniques related to delivery of simulated flavor sensations to a user while consuming (e.g., eating, drinking) food items. The perception of flavor is a multisensory experience as it involves not only taste, but other sensory experiences such as sight, sound, smell, and touch. Delivery of one or more sensory experiences to a user as the user is consuming a food or beverage item can augment how the user experiences the food or beverage item's flavor. For example, changing a smell or color of a food or beverage item effects how users perceive it during consumption. Changing perceptions through stimulating various senses can alter expectations significantly and in unexpected ways when delivering or combining stimuli. Accordingly, in some embodiments, the present disclosure provides for technologies (e.g., device(s), systems and/or method(s)) for delivering simulated flavor sensations while consuming food items (e.g., solid food items, a beverage).

In some embodiments, simulated flavor sensations are delivered to a user based on a target flavor profile through providing one or more sensory stimuli to the user. Typically, sensory stimuli are those that alter the perception of a user but do not affect food items through the addition of condiments and/or flavorings to the food items themselves. Rather, in accordance with various embodiments, sensory stimuli are delivered to users as they consume food items such that the user perceives a different in flavor based on the stimuli and the flavor profile provided.

Additionally, in certain embodiments, provided systems allow for the use of suitable sensory modules (e.g., a smell module, a thermal module, an electric taste module, a color projection module) for delivery of one or more simulated flavor sensations to a user. In certain embodiments, sensory modules may be used to stimulate different attributes of a flavor sensation (e.g. texture, fizziness) using an appropriate apparatus. In certain embodiments, a simulated flavor sensation may be, in part, based on images and/or video shown to a user.

Accordingly, by delivering one or more sensory stimuli to a user using the technologies disclosed herein, new design opportunities in user-flavor interactions are made available through modulating and/or overlaying sensory stimuli as a user consumes food and/or beverage items.

In one aspect, the disclosure encompasses systems for delivering a simulated flavor sensation to a user, the systems comprise one or more sensory modules, wherein the sensory modules are selected from the group comprising: a smell module for providing a scent corresponding to the simulated flavor sensation to the user and positioned to deliver the scent to the nose of the user; a thermal module for providing a thermal stimulus to a portion of the tongue and/or a portion of the upper lip and/or the lower lip of the user and/or a portion of an area between the upper lip and the nose of the user and/or one or more nerves (e.g., one or more cranial nerves) of the user; an electric taste module for electrically stimulating the tongue of the user; and/or a color projection module for providing visual cues to the user.

In some embodiments, the systems further comprise: a processor of a computing device; and a memory having instructions stored thereon, wherein the instructions, when executed by the processor, cause the processor to: receive and/or access data corresponding to the simulated flavor sensation; and use the data corresponding to the simulated flavor sensation to deliver the simulated flavor sensation to the user using at least one of the one or more sensory modules. In some embodiments, the instructions cause the processor to deliver an electric stimulus using the electric taste module to the tongue of the user. In some embodiments, the system comprises at least one electric taste module and the instructions cause the processor to deliver an electric stimulus corresponding to the simulated flavor sensation using the electric taste module to the tongue of the user. In some embodiments, the electric stimulus comprises a current from 0 to 200 μA. In some embodiments, the electric stimulus comprises a frequency from 0 to 1200 Hz.

In some embodiments, the systems further comprise: an image generating device for generating one or more images; and a communication module for transmitting the simulated flavor sensation corresponding to at least one of the one or more images to the processor. In some embodiments, the instructions, when executed by the processor, cause the processor to alter the data corresponding to the simulated flavor sensation based on the scene data. In some embodiments, the systems further comprise a structural frame adaptably configured to be mounted on the head of the user and wherein the image generating device is mounted to the structural frame for user observation of the video scenes.

In some embodiments, the electric taste module comprises: a set of electrodes; and a function generator operably connected to the set of electrodes. In some embodiments, the set of electrodes comprises at least two electrodes and wherein the electrodes are spaced apart by approximately 2 mm. In some embodiments, the set of electrodes comprises at least two electrodes and wherein the electrodes are spaced apart by 0.1 mm to 4 mm. In some embodiments, the function generator is and/or comprises a constant current source. In some embodiments, the set of electrodes is and/or comprises a conductive material. In some embodiments, the conductive material is and/or comprises a metal or a metal alloy. In some embodiments, the metal or metal alloy comprises at least 95% silver. In some embodiments, the conductive material comprises a conductive polymer.

In some embodiments, the smell module comprises one or more smell cartridges, wherein each of the smell cartridges comprises one or more compositions having a fragrance. In some embodiments, the fragrance comprises an essential oil. In some embodiments, the smell cartridge comprises a medium to absorb and/or contain the one or more compositions.

In some embodiments, the smell module further comprises one or more air-pumps and wherein each of the one or more smell cartridges is operably interfaced with at least one of the one or more air-pumps.

In some embodiments, the thermal module comprises a heating and/or cooling element. In some embodiments, the heating and/or cooling element is a thermoelectric heating and/or cooling element. In certain embodiments, the cooling and/or heating element is operatively interfaced with a thermally conductive material, wherein the thermally conductive material is in communicative contact with the portion of the tongue and/or the portion of the upper lip and/or the lower lip of the user and/or the portion of an area between the upper lip and the nose of the user and/or the one or more nerves (e.g., one or more cranial nerves) of the user. In certain embodiments, the cooling and/or heating element is operatively interfaced with a thermally conductive material, wherein the thermally conductive material is in communicative contact with the tongue of the user. In certain embodiments, the thermally conductive material is and/or comprises a metal. In certain embodiments, the thermally conductive material is 95% silver.

In some embodiments, the thermal stimulus is from 15° C. to 40° C.

In some embodiments, the thermal stimulus comprises rapidly heating and cooling from 18° C. to 38° C.

In some embodiments, the color module comprises one or more illumination sources.

In some embodiments, the color module comprises a diffusive element to disperse and/or soften the at least one of the one or more illumination sources.

In some embodiments, the systems comprise a vessel for containing a liquid.

In some embodiments, at least one of the one or more sensory modules are mounted to a vessel.

In some embodiments, the systems comprise an eating utensil for consuming a food item.

In some embodiments, the one or more sensory modules further comprises one or more additional modules, wherein the one or more additional modules stimulate different attributes of a flavor sensation.

In some embodiments, the one or more sensory modules further comprises one or more additional modules, wherein the one or more additional modules stimulate different attributes of a flavor sensation.

In some embodiments, one or more sensory modules may stimulate (e.g., directly or indirectly) one or more nerves of a user. For example, in some embodiments, the one or more nerves of the user comprises one or more cranial nerves. In some embodiments, the one or more cranial nerves is or comprises a trigeminal nerve.

In another aspect, the disclosure is directed to methods of delivering a simulated flavor sensation to a user, the methods comprise: receiving a target flavor profile corresponding to a simulated flavor sensation; and stimulating the user based on at least the target flavor profile by performing one or more of the following: dispensing, to the nose of the subject, one or more fragrances corresponding to the target flavor profile; heating or cooling a portion of the tongue of the user and/or a portion of an upper lip of the user and/or a portion of an area between the upper lip and nose and/or one or more nerves (e.g., one or more cranial nerves) of the user using a thermal module, wherein a temperature of the thermal module corresponds to the target flavor profile; electrically stimulating the tongue of the user corresponding to the target flavor profile; and/or projecting one or more colors of light corresponding to the target flavor profile.

In some embodiments, the step of stimulating the user further comprises stimulating the user based on one or more images shown to the user.

In some embodiments, electrically stimulating the tongue of the user comprises delivering an electrical signal to the tongue of the user. In some embodiments, the electrical signal comprises a current from 0 to 200 μA. In some embodiments, the electrical signal comprises a frequency from 0 to 1200 Hz. In some embodiments, the electrical signal corresponds to a bitter taste. In some embodiments, the electrical signal corresponds to a sour taste. In some embodiments, the electrical signal corresponds to a salty taste.

In some embodiments, dispensing the one or more fragrances comprises blowing at least one of the one or more fragrances to the nose of the user.

In some embodiments, dispensing the one or more fragrances further comprises pacing the dispensing of the one or more fragrances.

In some embodiments, the heating or cooling comprises heating or cooling a heating and/or cooling element to a temperature from 15° C. to 40° C. In some embodiments, the heating or cooling corresponds to a sweet taste and comprises rapidly heating and cooling from about 18° C. to about 38° C.

In some embodiments, the methods comprise updating the target flavor profile based on a region in which the user resides and/or originates.

In some embodiments, the methods comprise updating the target flavor profile based on a scenario presented to the user.

In some embodiments, the methods comprise updating the target flavor profile based on one or more pre-calibrated profiles for the user.

In some embodiments, the one or more nerves of the user comprises one or more cranial nerves. In some embodiments, the one or more cranial nerves is or comprises a trigeminal nerve.

In another aspect, the disclosure encompasses systems for delivering a simulated flavor sensation to a user, the systems comprising one or more sensory modules, wherein the sensory modules comprise: a smell module for providing a scent corresponding to the simulated flavor sensation to the user and positioned to deliver the scent to the nose of the user; and a thermal module for providing a thermal stimulus to a portion of the tongue and/or a portion of the upper lip and/or the lower lip of the user and/or a portion of an area between the upper lip and the nose of the user and/or one or more nerves of the user.

In another aspect, the disclosure encompasses systems for delivering a simulated flavor sensation to a user, the systems comprising one or more sensory modules wherein the sensory modules comprise: a smell module for providing a scent corresponding to the simulated flavor sensation to the user and positioned to deliver the scent to the nose of the user; an electric taste module for electrically stimulating the tongue of the user; and a color projection module for providing visual cues to the user.

In another aspect, the disclosure encompasses methods of delivering a simulated flavor sensation to a user, the methods comprising: receiving a target flavor profile corresponding to a simulated flavor sensation; and stimulating the user based on at least the target flavor profile by: dispensing, to the nose of the subject, one or more fragrances corresponding to the target flavor profile; and heating or cooling a portion of the tongue of the user and/or a portion of an upper lip of the user and/or a portion of an area between the upper lip and nose and/or one or more nerves using a thermal module, wherein a temperature of the thermal module corresponds to the target flavor profile.

In another aspect, the disclosure encompasses methods of delivering a simulated flavor sensation to a user, the methods comprising: receiving a target flavor profile corresponding to a simulated flavor sensation; and stimulating the user based on at least the target flavor profile by: dispensing, to the nose of the subject, one or more fragrances corresponding to the target flavor profile; electrically stimulating the tongue of the user corresponding to the target flavor profile; and/or projecting one or more colors of light corresponding to the target flavor profile.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIGS. 1A and 1B are images of two exemplary virtual flavor apparatuses, according to illustrative embodiments.

FIG. 2A is a schematic of an exemplary virtual coffee device, according to an illustrative embodiment.

FIG. 2B is a schematic of an exemplary virtual cocktail device, according to an illustrative embodiment.

FIG. 3A is an image of a subject interacting with an exemplary virtual coffee device, according to an illustrative embodiment.

FIG. 3B is an image of a subject interacting with an exemplary virtual cocktail device, according to an illustrative embodiment.

FIG. 4 is graph of average intensity scores of different stimuli from a virtual beverage device.

FIG. 5 is graph of average intensity scores of different stimuli from a virtual coffee device.

FIG. 6 is graph of average liking scores of different stimuli from a virtual beverage device.

FIG. 7 is graph of average liking scores of different stimuli from a virtual coffee device.

FIG. 8 is a series of graphs showing average intensity scores of different stimuli combinations in terms of primary taste sensations reported from the virtual beverage glass.

FIG. 9 is a series of graphs showing average liking scores of different stimuli combinations using virtual beverage glass.

FIG. 10 is graph of emotions mediated by different virtual flavor stimuli from a virtual beverage device.

FIG. 11 is graph of emotions mediated by different virtual flavor stimuli from a virtual coffee device.

FIG. 12 is a block diagram of an exemplary cloud computing environment, used in certain embodiments.

FIG. 13 is a block diagram of an example computing device and an example mobile computing device used in certain embodiments.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Definitions

About: The terms “about” or “approximately”, when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the terms “about” or “approximately” may encompass a range of values that within 25% or less of the referred value.

### DETAILED DESCRIPTION

It is contemplated that systems, architectures, devices, methods, and processes of the claimed invention encompass variations and adaptations developed using information from the embodiments described herein, as may occur to those of skill in the art.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously, except where otherwise specified.

The mention herein of any publication is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

Presented herein are compositions, systems, and methods related to the delivery of one or more flavor sensations to a user. In certain embodiments, provided technology relates to systems for delivery of simulated flavor sensations to a user (e.g., a human, an animal, e.g., a cat, a dog) through stimulating the user via one or more modules (e.g., sensory modules), for example, as described herein.

Flavor is a multimodal sensation that is determined, inter alia, by taste and smell. Additional modalities including, but not limited to, color, texture, temperature, and sound also contribute towards the perception of flavor sensations. In some instances, flavors in foods and beverages evoke vivid, colorful memories and emotions. Additionally, there can be close associations of human olfaction and gustation with memory and emotional responses. Due to this entwined nature of flavor perception and emotional responses, flavors can remind individuals of, for example, past positive or negative memories such as a joyful holiday, being ill, or other. For example, some individuals prefer to have a cup of tea or coffee when tired, eat candies when stressed, or sip a glass of lemonade on a hot summer day to relax. All of these experiences are related to emotions. Accordingly, individual application or co-application of stimuli that appeal to one or more of senses through the technology disclosed herein can have significant and surprising effects on what flavor the user perceives or is experiencing as they consume a food or beverage.

As explained herein, the tastes of foods or beverages are conventionally altered to a user's individual taste through the addition of condiments or other additives. For example, coffee can be made sweeter by the addition of sugar. Additionally or alternatively, changing the roast or source of the coffee beans can significantly alter flavor. However, these changes are often permanent. For example, once sugar is added it cannot be practically removed, and the coffee is potentially ruined for a particular individual by adding too much. In addition, sugars and other condiments can be detrimental to the health of users. For example, often users on restricted diets such as diabetics or those with obesity are advised to not consume beverages that have high amounts of sugar. Against medical advice, a user may continue using these condiments and/or additives. Alternatively, users are able to seek out low calorie alternatives, but often these are unsatisfying.

Accordingly in certain embodiments, technologies discussed herein affect flavors of food items without having a permanent effect on the food or beverage item and/or without a detrimental health effect (e.g., physical and/or dietary in nature) on the user. By providing a simulated flavor sensation to a user consuming a food item, flavors can be augmented and/or enhanced to the user's liking without additional condiments such as sugar. In some embodiments, provided technologies can also be used to allow a user with one or more impaired modalities (e.g., taste or smell) to experience flavors that were previously unperceivable, whether due to illness or injury.

For example, in certain embodiments, a simulated flavor sensation is delivered to a user through use of one or more target flavor profiles. A target flavor profile can have information regarding tastes and/or parameters the user particularly desires. For example, a user drinking unflavored water may want to instead experience a coffee beverage. A simulated flavor sensation then would deliver a target flavor profile corresponding to the sweet coffee beverage to the user using one or more stimuli. For example, the target flavor profile could have heat and/or smell information based on the information related to the simulated coffee flavor. An appropriate amount of heat would then be provided to the user's nasal area to simulate coffee's heat. In addition, a coffee smell can be delivered to the nose of the user as they drink. Such a combination of stimuli would provide an experience that was like drinking coffee, yet provides no calories or permanent changes to the water being consumed by the user. Exemplary systems and methods for creating such stimuli are further described herein. In certain embodiments, drinks other than water (e.g., a base drink) could be used. In certain embodiments, digital flavorings can also be used to mask an undesirable taste of a food item (e.g., too bitter). In some embodiments, the use of such masking could be used, in whole or in part, to hide the taste or other sensation of one or more ingredients that are healthy for a user, but for which the user dislikes the taste or other attribute of the ingredient (e.g., the taste or texture of a protein or kale powder, etc.).

In addition, neither the concept of flavor nor its association with human emotions have been well studied and/or utilized in Virtual Reality (VR), Augmented Reality (AR), and Human-Computer Interaction (HCI) domains. In particular, fundamental concepts such as the contribution of various sensory modalities, including, but not limited to, taste, smell, color, and temperature (e.g., heat/cool), towards flavor perception and the human emotions mediated through flavor sensations remained underexplored in the domains mentioned above. In certain embodiments, the provided technology can be used to determine how to effectively integrate the concept of flavor into the process of digital interaction design to open up new design opportunities in VR and Human-Flavor Interaction. Accordingly, in certain embodiments the technology described herein integrates computational methods and sensory stimulations in order to alter flavors of food items experienced by a user.

### Sensory Modules

In certain embodiments of the technology as described herein, any application appropriate sensory module(s) may be used. Sensory modules are used to stimulate one or more senses of a user, including but not limited to sight, touch, smell, taste, and sound. Alternatively or additionally, multiple sensory modules may be used to stimulate the same sense or senses.

Stimuli delivered by the technology described herein may be used to provide a simulated flavor sensation to the user by stimulating one or more senses (e.g., one or more nerves, e.g., one or more cranial nerves) of the user as the user consumes a food product such as a drink or a solid food item.

In certain embodiments as described herein, sensory modules do not cause a permanent change in the flavor of the food item itself. For example, once the stimulus is stopped, taste of a food item perceived by a user is returned to the state prior to the application of the stimulus within a period of time (e.g., a minute, five minutes, fifteen minutes, a half hour). This feature provides a significant benefit over, for example, chemical-based flavorings (e.g., sugars, salts, synthetic flavorings) that are traditionally applied to food items in order to enhance and/or augment flavor. Unlike traditional chemical-based flavorings, sensory modules described herein can be dynamically adjusted and/or reset whenever needed based on a given momentary desire. Additionally, stimulating a sensation rather than adding a flavoring is a low-calorie option to enhancing/changing flavors quickly based on individual desires.

In certain embodiments, one or more sensory modules can be mounted and/or incorporated into any application appropriate eating utensil or vessel used for consuming a food or beverage item. For example, in certain embodiments eating utensils may include, without limitation, a fork, a spoon, a knife, a cup, a glass, or chopsticks. In certain embodiments, modules can be mounted onto a structural frame, which can be detached from the eating utensil. How modules are positioned on utensils would be understood by those of skill in the art based on the descriptions provided herein.

Described in the below section are a selection of exemplified sensory modules. These exemplified modules are not intended to limit the technology, but to provide examples of modules and/or methods that may be incorporated into the technology described herein.

### Smell Module

In certain embodiments of the technology as described herein, any application appropriate method or system(s) may be incorporated in order to provide a scent (e.g., a smell) corresponding to a simulated flavor sensation to a user. In certain embodiments, this may be done through a dedicated module (e.g., a “smell module”) or through incorporation of application appropriate technology such as technologies disclosed herein in another component or module. In some embodiments, a smell module may provide a simulated flavor sensation through olfactory stimulation (e.g., direct or indirect stimulation) of one or more nerves (e.g., cranial nerves, e.g., one or more olfactory nerves).

Scents contribute significantly towards perception of flavors and/or taste sensations of food items. In certain embodiments, co-exposure of taste sensations (e.g., primary taste sensations of salty, sour, sweet, bitter, and umami) together with one or more scents generally provides a wholesome sensory experience of the flavor. For example, matching scent-taste pairs increases perception of certain flavors. In one such example, pairing a sour taste and lemon smell increases perception of a sour flavor or sensation. In contrast, non-matching scent-taste pairs surprisingly increase perception of several different taste sensations. In another example, pairing a sour taste with chocolate smell increases perception of salty, sour, and sweet sensations.

A smell module as described herein can be implemented in order to deliver scents (e.g., fragrances, aromas) to a user via an orthonasal and/or retronasal olfaction approach. That is, in certain embodiments the technology as disclosed herein can deliver scents to the nose of a user and/or through the oral cavity of the user.

Fragrances delivered to a user using smell modules may be derived from any suitable scented composition. Given the importance of user safety during delivery of scents, compositions having food grade and/or food safe qualities is desirable. Food grade qualities include being safe for human and/or animal consumption as well as being safe for contact with food. This may include not causing adverse and/or harmful reactions to the user and/or the technology. Examples of suitable compositions that would provide a fragrance includes, but is not limited to, essential oils, alcohol (e.g., wine), spirits, and fragrant esters (e.g., isoamyl acetate). Many artificial scents have, contain, and/or are compositions which react with foods, and may not be suitable for use under certain conditions.

The technology disclosed herein may encapsulate fragrances in any suitable container (e.g., a cartridge). In certain embodiments, fragrances may be from a mixture of compounds contained in a smell container. Additionally, the technology may incorporate multiple smell containers into a single device. For example, smell containers can be directly incorporated into a utensil and/or device to consume food (e.g., a bowl, a cup). A smell container may also include a medium (e.g., a cotton ball, gel, or liquid) which may be imbued with (e.g., by soaking or through manufacturing techniques) a fragrance. In some embodiments, a stimulus (e.g., an electronic signal, heat, or the like) may be applied in order to generate a scent from a smell module. For example, a stimulus (e.g., heat) may be used to heat an oil or other scented composition. In some embodiments, allowing for simple diffusion of a scented composition may be sufficient to provide a scent to a user.

In certain embodiments, smell containers are connected to a pump or device which can create a pressure gradient to cause smells to be delivered to a user. For example, micro-air pumps may be used to push air into a smell container and carry a fragrance to the nose of the user. In addition or alternatively, ultrasound atomizer devices (similar to those used in aroma therapy and other applications) may also be used to aerosolize fragrances for delivery to a user.

In certain embodiments, delivery of the fragrance to the nose of the user is, in certain embodiments, controlled by an algorithm. For example, an algorithm may be used to control the rate and/or volumes at which the fragrance is dispersed to the user. Algorithmic control using a pulse width modulation (PWM) technique is important to control the intensity of a smell the user experiences. Furthermore, an algorithm allows a user to adjust different smells or fragrances in order to find a desirable mixture of smells from smell containers.

### Thermal Module

In certain embodiments of the technology as described herein, any application appropriate method or system(s) may be incorporated in order to heat and/or cool a user [e.g., a user's lip or other local area, the user's tongue, and/or one or more nerves of the user (e.g., one or more cranial nerves)] according to a corresponding simulated flavor. In certain embodiments, this may be done through a dedicated module (e.g., a “thermal module”) or through incorporation of application appropriate technology such as those disclosed herein in another component or module. In some embodiments, a thermal module may provide a simulated flavor sensation through stimulation (e.g., direct or indirect stimulation) of one or more nerves (e.g., one or more cranial nerves, e.g., one or more trigeminal nerves).

In some embodiments, a thermal module may include components which come into contact with a portion of the upper lip and/or lower lip of the user. For example, altering flavors of a beverage can be done by applying thermal sensations on the skin around the nose simulates skin temperature changes when drinking a beverage. Without wishing to be bound to any particular theory, flavor richness and aftertaste are significantly enhanced by heating the upper lip and nasal region of the user.

In certain embodiments, one or more components, for example, of a provided system, may also or alternatively come into contact with an area between the upper lip and the nose of the user. For example, in certain embodiments the systems and methods disclosed by Suzuki et al. (Affecting tumbler: affecting our flavor perception with thermal feedback, in In Proceedings of the 11th Conference on Advances in Computer Entertainment Technology, p. 19. ACM, 2014.), which is incorporated by reference with regards to the heating apparatus used to provide heat to the user, may be utilized to apply thermal sensations onto or around the skin near the nose of the user to simulate skin temperature change.

In addition or alternatively, components of the thermal module may come into contact with a portion of the tongue of the user. In particular, components of the thermal module may come into contact with the tip of the tongue to heat and/or cool the user's tongue. Heating and/or cooling the tongue of the user can create a thermal sensation that also has an effect on taste. For example, cooling the tip of a user's tongue to about 18° C. can create a mint and/or menthol sensation. In another example, heating the tip of a user's tongue to about 38° C. can create a hot (e.g., spicy) sensation.

A thermal module can be comprised of any suitable heating and/or cooling element that can be used to rapidly heat and/or cool. In certain embodiments, the heating and/or cooling element maintains a portable profile. For example, a thermoelectric heating/cooling device such as a Peltier device is used in some embodiments to provide for heating/cooling of the skin. In certain embodiments, the heating and/or cooling element can be attached to a conductive material so that the heating element itself does not directly come into contact with the user.

In certain embodiments, a thermoelectric heater/cooler can be attached to a thermally conductive material that comes into contact with the user (e.g., the tongue of the user, the upper/lower lip of the user, an area between the nose and upper lip of the user). The thermally conductive material can be any suitable material that allows for the transfer of heat/cold sensations. For example, metals or metal plated materials are thermally conductive and allow for heat to be efficiently transferred to the user. Food-safe conductive metals or metal alloys, such as those containing silver, gold, platinum, or palladium, are desirable. In certain particular embodiments, metals containing 95% silver are desirable for their food safety and relatively low price. Additionally or alternatively, the conductive materials or heating elements may be embedded within a ceramic in order to provide a protective barrier between the user and the conductive material and/or heating element, thus enhancing user safety. In certain embodiments, the conductive materials may be thermally conductive plastic.

In certain embodiments, the thermal module is used to create and/or augment particular flavor perceptions by heating and/or cooling a surface of the body with which the components of the thermal module come into contact. For example, by rapidly heating a portion of the tongue (e.g., the tip of the tongue) of a user, from 18° C. to 38° C. (e.g., at a rate of approximately 2° C. per second), and then cooling down to about 18° C. (e.g., at a rate of approximately 2° C. per second), users have reported perceptions of a sweet flavor during the hot to cold transition. Additionally, it has been found that minty or menthol flavors can be created by cooling down the tip of the tongue to a temperature below 20° C. (e.g. about 18° C. or lower). Furthermore, a spicy or hot flavor can be created by heating up the tip of the tongue to a temperature over 35° C. (e.g., about 38° C.).

For safety and comfort of users, in accordance with various embodiments, the temperature created on the body surface by the thermal module may be limited to about 15° C. and to about 40° C.

### Electric Taste Module

In certain embodiments of the technology as described herein, any application appropriate method or system(s) may be incorporated in order to electrically stimulate the tongue of a user (e.g., using an electric stimulus) according to a corresponding simulated flavor profile. In certain embodiments, this may be done through a dedicated module (e.g., an “electric taste module”) or through incorporation of application appropriate technology such as those disclosed herein in another component or module. In some embodiments, an electric taste module may provide a simulated flavor sensation through stimulation (e.g., direct or indirect stimulation) of one or more nerves (e.g., one or more cranial nerves, e.g., one or more facial nerves, one or more glossopharyngeal nerves, one or more vagus nerves). In certain embodiments, the stimulated nerves are those responsible for taste sensation.

In certain embodiments, an electric taste module has one (or more) function generators with which to deliver an electrical stimulus to either the upper surface or lower surface of the tongue of a user. The placement of the stimulus depends on the desired effect. The function generator delivers a stimulus when the tongue of the user comes into contact with a set of electrodes, which are arranged on a surface. For example, in certain embodiments, the set of electrodes may be arranged on, for example, a rim of a cup or bowl or incorporated into chopsticks as disclosed in Ranasinghe, N. et al., Food Research International (2018), which is hereby incorporated by reference in its entirety.

In some embodiments, electrodes are arranged on the surface, for example, of a food or beverage vessel, such that they are spaced apart by a gap. A distance of a gap between electrodes and/or an arrangement of electrodes should allow for at least two of the electrodes to come into contact with the tongue. However, in some embodiments, electrodes are separated by a gap of approximately 2 mm (e.g., between 0.1 and 4 mm). A gap between about 0.1 mm to about 0.4 mm may be particularly beneficial in that electrodes separated by this distance allow for both electrodes come into proper contact with the tongue. This arrangement also minimizes current flow through the tongue. Contacting the tongue of the user with a set of electrodes creates an electrical connection across the electrodes as electricity flows across the tissue of the tongue due to a completion of a circuit across the set of electrodes. Too narrow of a gap (e.g., less than 0.1 mm) may lead to pain as a consequence of high current density.

In some embodiments, the size (e.g., width, length) and/or the shape of the electrodes that comes into contact with a user is an important design consideration. For example, if the width of an electrode is too small (e.g., 1 mm or less), a user can experience pain and/or discomfort from an electrical stimulus. The pain and/or discomfort the user experiences is due to a high current density being created at a point of contact of the electrode with the tongue. In certain embodiments, a set of electrodes has an electrode acting as an anode and an electrode acting as an cathode as defined by the directionality of the flow of current. In certain embodiments (e.g., when using a constant current), an electrode acting as a cathode and an electrode acting as an anode do not change their identity as a cathode and anode. The size and/or shape of an electrode which acts as an anode has been found to be important. In certain embodiments, an electrode may have a round, diamond, square, or other similar shape.

In some embodiments, placement and/or location of the electrodes that contact the tongue is an important design consideration. For example, in some embodiments placement of electrodes can be on the same or opposite surfaces of the tongue. For example, two or more electrodes (e.g., a cathode and an anode) may contact the top surface of the tongue. In another embodiment, two or more electrodes (e.g., a cathode and an anode) may both contact the bottom surface of the tongue. In another embodiment, two or more electrodes (e.g., a cathode and an anode) may be located on opposite surfaces of the tongue.

The electrodes be made of any suitable material(s) that allow for electrical and/or thermal conductivity. It is also desirable for the material to be food safe for the safety of the user. In certain embodiments, metals, metal alloys, and/or metal-plated materials may be used to fabricate electrodes including, but not limited to, silver, gold, platinum, and palladium. In some embodiments, silver is particularly advantageous as it is inexpensive and widely available. In certain embodiments, the conductive metal or metal alloy may contain at least 95% silver. In other embodiments, conductive polymers and/or plastics of food grade quality may be used as electrodes.

In certain embodiments, the electrodes should not be made of materials that would react with body fluids (e.g., saliva). For example, copper is not a desirable material as it corrodes when in contact with saliva. Accordingly, use of copper, in some embodiments, could present a significant safety issue as materials would corrode with use over time and release potentially harmful by-products that could injure users.

In certain embodiments, the electrodes should not elicit chemical reactions in the food item being consumed. For example, applying an electric current through isotonic drinks could elicit chemical reactions (known as electrolysis), which could be harmful to users, or alter the flavor profile of a food or beverage, under certain conditions.

In some embodiments, the form and/or kind of electrical stimulation delivered to the tongue of the user can significantly affect a perceived flavor. In certain embodiments, a “constant-current” is delivered to the tongue of the user. Using a constant current can have advantages over other signal types in that it ensures that the same signal is being delivered across the tongue no matter the resistance or the impedance of the tissue. Generally, a current delivered as part of the electrical stimulation ranges from 0 μA to approximately 200 μA. In some embodiments, currents higher than 300 μA may induce pain and/or discomfort for users. In addition, in accordance with various embodiments, the frequency of the electrical stimulation ranges from approximately 0 to 1200 Hz. Frequencies greater than 1000 Hz are not generally used as human tissues are unable to distinguish frequency differences in this range. These signals can be modified and modulated in any suitable manner to deliver a taste as part of a target flavor profile.

In accordance with various embodiments, any of a variety of current—frequency combinations, along with electrode placement, can be used together to deliver a taste to a user. Varying the currency-frequency combinations may result in a user experiencing a corresponding change in taste. For example, a salty taste corresponds to approximately 20-50 μA and approximately 200-400 Hz (e.g., a current of about 40 μA and a frequency of about 400 Hz). In another example, a sour taste corresponds to approximately 140-180 μA and approximately 800-1200 Hz (e.g., a current of about 180 μA and a frequency of about 800 Hz). In a third example, a bitter taste corresponds to approximately 60-100 μA and 400-600 Hz (e.g., a current of about 80 μA and a frequency of about 600 Hz). A bitter taste may be further induced through application of an electrical stimulation to both the underside and the top surface of the tongue. For example, in certain embodiments, an anode electrode would be in contact with the underside of the tongue and a cathode electrode would be in contact with the top surface of the tongue. In certain embodiments, the current—frequency combinations may need to be further adjusted (e.g., dynamically) based on individual user experiences as it is possible for certain users to experience slightly different tastes for a particular current—frequency combination based on their taste preferences.

### Color Projection Module

In certain embodiments of the technology as described herein, any application appropriate method or system(s) may be incorporated in order to provide a visual stimulation to a user according to a corresponding simulated flavor profile. In certain embodiments, this may be done through a dedicated module (e.g., a “color projection module”) or through incorporation of application appropriate technology such as that disclosed herein in another component or module. In some embodiments, a color projection module may provide a simulated flavor sensation through projecting colored light and stimulating (e.g., directly or indirectly stimulating) one or more nerves (e.g., one or more cranial nerves, e.g., one or more optic nerves). For example, a color projection module may stimulate optic nerves by projecting colored light to eyes of the user.

Visual representations or colors of a food or beverage can play a significant role in influencing flavor sensations. Due to cross-modal interactions between colors and previous experiences with food and/or beverages, users sometimes create a pre-perception of flavor in their mind based on the color or a food and/or beverage. For example, yellow and green are commonly linked to sour sensations while red is linked to sweet sensations. Furthermore, when the food or the beverage has a bright color, flavors are perceived to be more intense. In contrast, users are often confused when they taste colorless solutions with different flavors due to the lack of color information. Accordingly, a color projection module providing color to a user through, for example, projecting color onto a food or beverage helps in augmenting the user's perception of a food's flavor.

In certain embodiments, LEDs or other color projecting technologies may be used to illuminate a user's food item to change its color. In certain embodiments, wireless LEDs (light emitting diode) modules such as discussed in Narumi et al. (Evaluating cross sensory perception of superimposing virtual color onto real drink: toward realization of pseudo-gustatory displays. In Proceedings of the 1st Augmented Human International Conference, p. 18. ACM, 2010), which is incorporated by reference in its entirety, may be incorporated into the technology in order to project a color into a beverage. In certain embodiments, LEDs (light emitting diode) modules such as discussed in Ranasinghe et al. (Vocktail: A Virtual Cocktail for Pairing Digital Taste, Smell, and Color Sensations. In MM '17: Proceedings of the 25th ACM international conference on Multimedia. October 2017. Pg. 1139-1147), which is incorporated by reference in its entirety, may be incorporated into the technology in order to project a color into a beverage.

### Additional/Alternative Modules

In certain embodiments of the technology as described herein, any application appropriate method or system(s) may be incorporated in order to simulate additional and/or different attributes of a flavor sensation.

In certain embodiments, modules that alter or provide for, without limitation, textures and fizziness, may be incorporated such that a user will perceive differences in taste. For example, a micro-vibration module can be incorporated to produce and/or maintain the fizziness or bubbliness of a carbonated beverage.

In certain embodiments, modules that provide sound to a user may be incorporated. Sound may also have an effect on what a user tastes or experiences while consuming a food item. For example, providing a “crunching” sound influences the perceived hardness of the food item being consumed.

### Virtual/Augmented Reality

In certain embodiments of the technology as described herein, any application appropriate method or system(s) may be incorporated in order to provide a video and/or audio experience for the user as they consume a food item. For example, in certain embodiments a user consuming food or a beverage may be provided a screen such as one found in a TV. In certain embodiments, the screen may be in a VR system and mounted to the user's head using a structural frame or any other application appropriate mechanisms that allow for the user to be stimulated by the visual experience as they consume food and/or a beverage.

The simulated flavor being delivered to the user may be synchronized or based in part on images or video being and shown to a user. In certain embodiments, changes in video or images being overlaid (similar to Augmented Reality scenarios) could result in changes in the corresponding simulated flavor sensation (e.g., through changing one or more particular flavor profiles being provided to the user.) For example, in one embodiment the look and texture and/or mouth-feel of a black coffee could be altered into a milk coffee. In another example, broccoli on a plate could be sensed as chocolate pieces while being consumed by the user. Information regarding changes in the flavor sensation and/or a new flavor profile could then be transmitted to the modules, devices, or systems of the technology. Information transmission can be accomplished using a communication module wirelessly using any suitable wireless means (e.g., WiFi, Bluetooth®, NFC, or similar technologies). Alternatively or additionally, information can be transmitted using any suitable wired connections.

For example, a user consuming a coffee beverage may be shown images of a lavender field. The simulated flavor profile may shift as the user drinks the coffee, where shifts in wind cause smells of lavender and/or coffee to be delivered to the user.

### Safety and Security Features (Software/Hardware)

In certain embodiments, the technology described herein may incorporate any suitable method or system(s) as security measures in order to provide for device and/or user safety. Exemplary security measures include, but are not limited to, hardware and/or software level implementations. Security measures may be incorporated into the technology either directly into a module (e.g., via instructions on memory) and/or be incorporated through one or more separate safety modules.

In some embodiments, hardware (e.g., low level—level 1) implementations of security may be used, for example, in one or more sensory modules. In certain embodiments, separate stimuli monitoring and/or control circuits (e.g., a safety module, e.g., an electrical stimuli safety module) continuously monitor different stimuli being delivered to a user. Separate circuits ensure that stimuli delivered to a user (e.g., using one or more modules) are within safety and/or comfort settings, for example, by limiting the strength and/or duration of a particular signal or set of signals. In certain embodiments, separate circuits continuously and/or intermittently monitor electric stimuli (e.g., current, voltage) and/or thermal stimuli being delivered to a user. In certain embodiments, separate circuits continuously and/or intermittently monitor intensity and/or duration of color stimuli (e.g., light output) delivered to a user. For example, hardware-based safety measures can be implemented that limit and/or define a maximum time each module can be activated (e.g., maximum 5 seconds for smell module at a time).

Software (e.g., high level—level 2) implementations of security measures may be incorporated into a device in any application-appropriate way, for example, through instructions provided on an individual module or multiple modules, and/or on a component of a system that is functionally connected to one or more modules. Software implementations can also be done through multiple modules depending on connectivity of the technology to other devices. Furthermore, additional software-based safety measures can be implemented such as defining a maximum time each module can be activated (e.g., maximum 5 seconds of activity for a smell module at a time).

In some embodiments, if a provided system or other device is connected to internet services, secure connections should be provided through suitable cryptographic and/or other connectivity protocols. For example, SSL (Secure Sockets Layer) or TLS (Transport Layer Security) protocols may be used to securely connect the devices, modules, and/or system(s) disclosed herein to a server. For example, in certain embodiments, a security certificate (e.g., a SSL certificate) is sent from a server to a device or system(s) as disclosed herein. Software on the device and/or system(s) verifies if the security certificate is able to be trusted. Then, the server is able to send a profile to the device and/or system(s) as it is a trusted source of information.

In certain embodiments where technology provided system or other device is connected to a mobile application (an “app”), software level implementations of security provide another level of security in addition to Bluetooth® or other suitable short range peer to peer communication protocols (e.g., near field communication (NFC), radio-frequency identification (RFID)).

In certain embodiments, a provided system or other device can include one or more additional security measures to monitor power distribution to different hardware modules (e.g., hardware and/or software-based measures). For example, in some embodiments, software may be used to monitor an electrical stimulus (e.g., a current, a voltage) delivered to a user.

### Target Flavor Profile and Updating Settings

In certain embodiments as described herein, a target flavor profile may be updated and/or changed based on experiences provided to a user.

For example, in certain embodiments, a target flavor profile may be updated based upon a region in which the user resides and/or originates. It is generally known that flavors are affected not only by hereditary genetic components, but also are affected by experiences as users age. For example, a user who has grown up eating heavily spiced foods may require more intense stimulations than an individual who has only experienced more bland foods. Furthermore, regular smokers (e.g., of tobacco, cannabis, and the like), subjects on restricted diets, subjects having a condition (e.g., cancer, Alzheimer's disease), or subjects undergoing medical treatment (e.g., chemotherapy) may also require more intense stimulations. Accordingly, the target flavor profile may require adjustments to one or more settings (e.g., a current-frequency combination of an electrical stimulation) in order to produce the desired flavor sensation to one user as opposed to another.

In certain embodiments, the target flavor profile can be updated based on one or more pre-calibrated profiles for a user. For example, given that a user has a particular background or likes and/or dislikes, the technology disclosed herein can incorporate any appropriate tuning mechanisms to either algorithmically and/or manually change the settings for stimulating a user.

In addition, not all users experience the same tastes with the same settings. For example, a user may experience a clear salty taste with higher intensity while using an electrical stimulation with a current of about 40 μA and a frequency of about 400 Hz. In contrast, a second user may experience a clear salty flavor at a current of about 45 μA and a frequency of about 410 Hz.

In certain embodiments, the target flavor profile can be adjusted based on the ingredients in food or beverages. For example, to enhance the saltiness of a low sodium soup one might need only a minor enhancement to achieve the same salty flavor as compared to a no sodium food (e.g., a no sodium soup).

Furthermore, in certain embodiments, a target flavor profile may be updated based on a particular scenario presented to a user. For example, having the ability to control food element digitally will enable chefs and bartenders to create extraordinary dining concepts and scenarios.

In one scenario, for instance, a diner will choose to drink black coffee before a meal and add chocolate flavor as a dessert coffee after a meal.

Additionally or alternatively, flavor profiles may be updated based on the time of day a user is consuming food. For example, consuming the flavor profile may provide a less sweet flavor experience in the morning in comparison to the evening. This will allow for user preferences to be customized without more complex inputs.

In certain embodiments, the target flavor profile can be updated using an application (e.g., an “app”) on a user's device (e.g., a computer, a cellphone). Users can update based on their own preferences for a particular flavor to “fine tune” flavors. In certain embodiments, target flavor profiles can be received from other users and further adjusted to a particular desired flavor.

Additionally, in some embodiments, flavor profiles can be dynamically changed as a user consumes a particular food or beverage. By way of example, in a fine dining setting, a flavor profile may be intentionally changed as a user consumes a particular dish or beverage to provide a varied consumption experience simply not possible without applying the present technology.

### Examples

### Exemplified Devices

This section provides a description of exemplary device configurations along with data demonstrating effects of different stimuli combinations on users' perception of flavors and/or emotions.

To study the effects of different sensory modalities on flavor perception, Applicant presents an approach to formulate flavor sensations by overlaying taste, smell, color, and thermal stimuli on plain water (i.e. Virtual Flavors). Furthermore, Applicant investigated the influences of these multimodal stimuli on perception of virtual flavors in terms of four primary taste sensations (salty, sweet, bitter, and sour). Applicant also examined how different modalities affect participants' emotions and liking towards a given simulated flavor sensation.

Surprisingly, it was found that participants did not need to be provided a flavor in the form of a food additive or even provided a stimulation on the tongue via a module in order to have their sense of taste altered. It was also found that by overlaying one or more smell sensations, participants perceived augmented taste sensations. For example, by overlaying a strawberry or vanilla scent, participants perceived an increase in the sweetness of plain water.

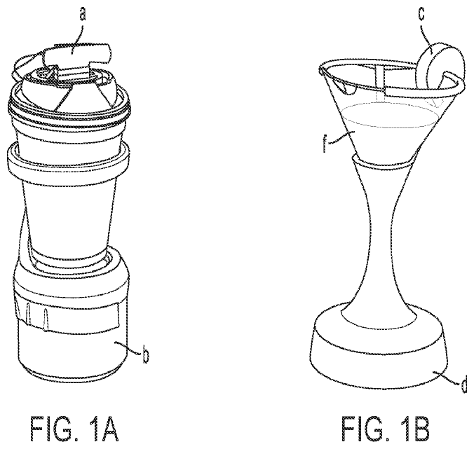

FIGS. 1A and 1B show two exemplary apparatuses used to deliver simulated flavor sensations.

In FIG. 1B, an exemplary virtual beverage glass is depicted. The virtual beverage glass manipulates taste by electrical stimulation (e.g., electrical stimulation on the tongue via silver electrodes attached on a rim of a glass), smell (e.g., dispersed on a surface of a beverage via micro air pumps and essential oils), and color (projected on the beverage via RGB Light Emitting Diodes). When a user drinks from the virtual beverage glass, colors are projected on to the beverage (visual), smells are released on the surface of the beverage (olfaction), and sour and salty sensations are applied via electrical stimulation (gustation) in order to simulate various flavor sensations.

The virtual beverage glass (FIG. 1B) was developed by attaching a martini glass onto a 3D printed structural base (d) that holds the electronic control module, three scent cartridges (e.g., containers), and three micro air-pumps. In principle, more or fewer scent cartridges and/or pumps may be used depending on the application. In this embodiment, (c) is a smell and color stimuli delivery module. An LED inside the module is positioned to illuminate the beverage. In addition, a tube inside of the module leads to the 3D printed base (d) of the virtual beverage glass. On the rim of the glass, two electrodes (f) are positioned with a gap between them so that they are likely to come into contact with the user's tongue.

In FIG. 1A, an exemplary virtual coffee mug is shown. The virtual coffee mug simulates an experience of drinking a coffee beverage based on smell (dispersed on the surface of the beverage via micro air pumps and essential oils) and thermal (via Peltier elements attached on the disposable coffee mug) sensations alone. In the virtual coffee mug, virtual flavors are delivered through scents dispersed near the user's nose (olfaction) and thermal stimuli (hot and/or cold temperatures) near the user's upper lips (to simulate temperatures of coffee). Users' perception of single or combined stimuli creates virtual flavor sensations not inherent to the plain water they consume while using the apparatus. The virtual coffee mug was developed by attaching a disposable paper coffee cup to a 3D printed structure that consists of a control module, three scent cartridges, and a Peltier element. The 3D printed structure is removable such that a new and/or different container is able to be inserted into the structure. The control module and scent cartridges are found in the base (b) of the 3D printed structure. The heated element (a) that comes into contact with the upper lip of the user is positioned on the lid of the cup.

Thus, the working principle is to overlay different, externally applied stimuli when drinking from these two apparatuses. The co-application of stimuli enables users to experience different virtual flavors. The virtual beverage glass is intended to simulate common beverages (e.g., soft drinks) while the virtual coffee mug is intended to simulate a coffee-like experience. Coffee is an emotionally recognized beverage all around the world, which makes it a particularly useful beverage for study. However, the device is not limited to simulation of coffee or any particular beverage.

Different sensory modalities were featured in two apparatuses to simulate virtual beverage experiences. In the virtual coffee mug, two sensory modalities, smell and temperature, were selected. The virtual beverage glass was integrated with the most common sensory modalities utilized in everyday mixed drinks—visual (e.g., light), smell, and taste.

A user experiment was conducted using both devices to apply different combinations of electric stimuli, smell, color, and thermal stimuli over plain water at room temperature. Participants were instructed to use both devices to consume virtually flavored water and provide their feedback on perceived flavor sensations (in terms of four primary taste sensations: salty, sour, bitter, and sweet), their liking towards simulated sensations, and emotions they related to these flavor stimuli. Experimental findings not only revealed that participants perceived different virtual flavor sensations with various stimuli configurations, but also had varied responses in terms of emotions and likeness towards simulated sensations. Furthermore, it was surprising that the combination of heat and smell stimuli were able to alter users' perception of the taste of water.

Liking towards certain virtual flavor stimuli and mediated emotions from virtual flavor stimuli remain unexplored in VR and HCI, especially when coupled with electric taste sensations. People's hedonic liking towards a flavor sensation is mainly based on its smell and taste sensory impressions. In addition, previous or repeated exposure, pleasant or unpleasant experience, memories related to a specific flavor sensation, and ambient noise are also some of the factors affecting the liking towards a flavor sensation. People also like certain flavors due to the number of calories associated with the food or drink. For example, although beverages such as beer and coffee taste bitter, people still develop higher preferences due to the calories consumed and smells released. This phenomenon is known as the ‘associative learning of flavors’ where the liking responses are increased when the taste sensations are paired with smell sensations. For example, in general, smells associated with sweet taste receive higher liking scores when compared to smells linked with bitter taste.

Flavors have a strong influence on people's emotions and vice versa. Flavors can regulate and alter our emotions, at the same time, emotions can influence our food choices and appetite. However, in VR and HCI research, mediated emotions through flavor sensations are not studied in detail, let alone the influence of different flavor attributes towards people's emotions.

Taste-emotional valence mapping (sweet taste as a positive emotion and bitter as negative emotion) extends into real-life scenarios, and the taste-emotional arousal mapping is proportional between each other (higher the taste higher the emotions). Measuring emotions in an interactive system is uniquely challenging as people's emotions vary based on multiple factors such as the context of use and difficulties in accurately verbalizing specific emotions. There exist many scales, such as Self-Assessment Manikin (SAM), Emotion wheel, and EsSense Profile, as well as a number of vocabulary terms to measure different emotional conditions. Applicant adopted the EsSense Profile to measure participants' emotions. The EsSense profile is widely accepted in measuring emotions associated with foods and beverages, especially in sensory sciences and studies. Thus, key contributions of this technology were examining a wide range of digital stimuli configurations and specific changes in flavor perception with regards to four primary taste sensations salty, sour, bitter, and sweet; studying participants' hedonic liking towards various stimuli configurations; studying the emotions mediated through different stimuli configurations; and introducing an approach to implement digital flavor experiences into AR and/or VR technologies. In addition, the work presented herein demonstrates digital controllability of flavor sensations.

### Technical Details of Exemplary Apparatuses

This section describes technical information on both apparatuses—a virtual beverage glass and a virtual coffee mug, that were used to deliver virtual flavor sensations. When a user took a sip from either of these apparatuses, smell, taste, color, and thermal stimuli was applied using technology described herein to enhance the flavors of the base drink (i.e., plain water). The virtual beverage glass had three subsystems as shown in FIG. 2B: 1) a color projection module, 2) an electric taste module, and 3) a smell module. The virtual coffee mug had two subsystems as illustrated in FIG. 2A: 1) a smell module and 2) a thermal module.

FIGS. 2A and B show different components of FIG. 2A a virtual coffee mug, and FIG. 2B a virtual beverage glass. Components of the devices are listed as follows: (a) micro-air pumps, (b) smell containers, (c) main control module with Bluno Nano, (d) temperature module with Peltier element, (e) disposable coffee cup, (f) RGB color module and smell diffusing tube, and (g) electric taste module. Different subsystems in both apparatuses were controlled by DFRobot's Arduino Bluno Nano 1 modules. Various combinations of stimuli could be formulated and activated using a computer or a smartphone via Bluetooth connection.

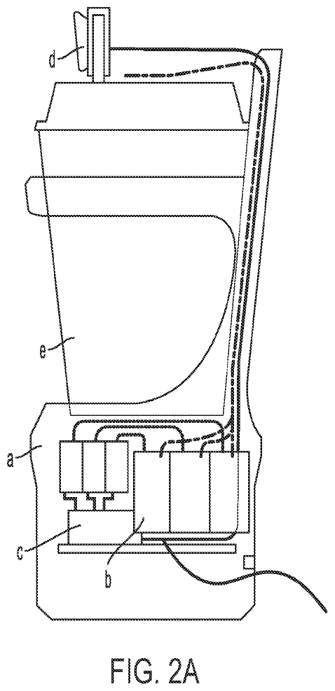

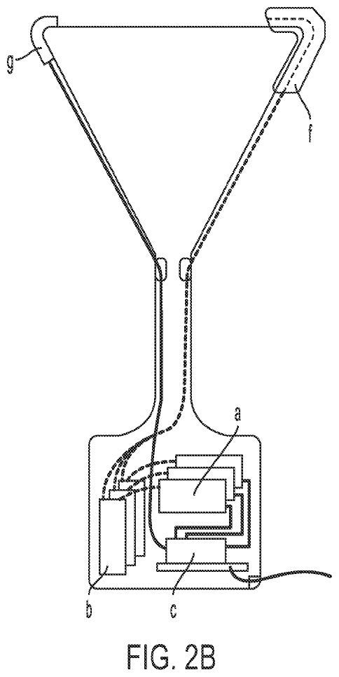

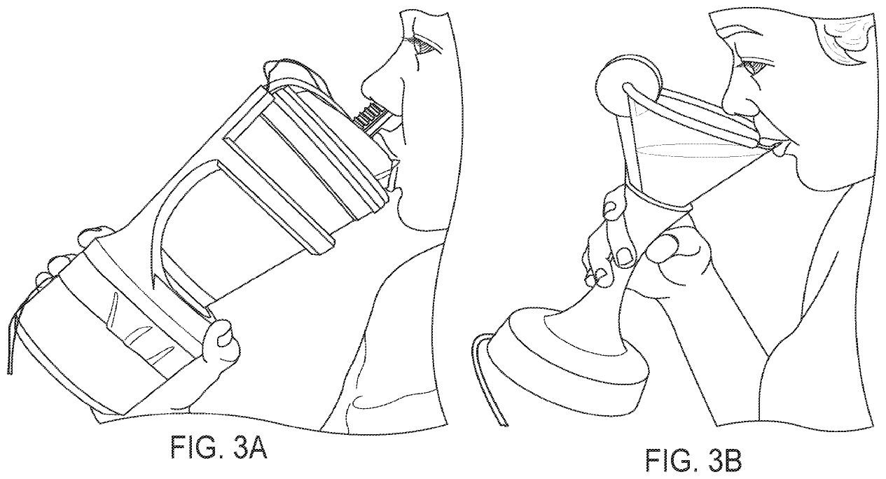

FIG. 3A shows an individual interacting with a virtual coffee cup device.

FIG. 3B shows an individual interacting with a virtual beverage glass device.

### Exemplary Color Projection Module

In this example, a color projection module was used to project a color to a user. An RGB LED was attached to the bottom surface of the virtual beverage glass. A light-diffusing layer was provided by a white-colored, diffuse-paper to enhance illumination of the beverage. Based on different stimuli configuration, distinct colors could be projected on to the beverage.

### Exemplary Electric Taste Module

In this Example, a constant-current source was developed in the virtual beverage glass to apply weak and controlled electrical pulses to the user's tongue while drinking, thus simulating, for example, sourness and saltiness. The constant-current source ensured that the electrical stimuli were not altered depending on the resistance of the user's tongue. Electrical stimuli were presented via two 95% silver electrodes attached to the rim of the virtual beverage glass. Electric sour and salty stimuli were formed as listed below:
Sour: magnitude of current: 180 mA; frequency: 800 Hz
Salty: magnitude of current: 40 mA; frequency: 400 Hz

### Exemplary Smell Modules

In this example, the smell modules in both apparatuses were implemented using three components: micro air-pumps, smell containers that hold essential oils, and polyurethane tubes. Each air-pump could be actuated individually, forcing air into a specific smell container. Each smell container had essential oil-soaked cotton balls that acted as a medium to hold the particular scent consistently.

In the virtual beverage glass, lemon and chocolate scents, as well as a spirit-like scent (prepared by mixing lime, cognac, and isopropyl alcohol), were used. Coffee, chocolate, and mint essential oils are used in the virtual coffee mug. The orthonasal olfaction approach was employed to deliver a smell stimulus to the user's nose. The intensity of a smell stimulus was regulated using a Pulse-Width Modulated (PWM) control algorithm to operate air-pumps in specific paces. Essential oils were selected from Aftelier Chef's Essence® flavor products.

### Exemplary Thermal Module

In an exemplary virtual coffee mug, a Peltier element attached to a silver metal piece was used to apply controlled thermal stimulus on or near the user's upper lips simulating hot or cold coffee experiences. A Pulse-Width Modulated (PWM) control algorithm was implemented to manage the intensity of the thermal stimulus. The direction of the Peltier element (hot vs. cold) was controlled via a motor driver, which consumed approximately 7 Watts. A heat sink was attached to the Peltier module to extract excessive heat generated by the Peltier element over time. Additional safety mechanisms (e.g., software-based safety mechanisms) were built into the thermal algorithm to make sure the thermal stimulus applied was within a comfortable range.

### Experimental Setup and Evaluation of Exemplified Embodiments

Described herein are experimental results relating to a study conducted using exemplified embodiments of the technology as disclosed herein. The primary purposes of the experiments presented and discussed herein were to (1) validate and support the concept of virtual flavor technology and (2) determine the influence of stimuli on participants' emotional profile while consuming different simulated beverages. A goal was that participants would be able to perceive and like different beverages based on different stimuli configurations. It was also hypothesized that emotions of the participants would be influenced by the different stimuli combinations that will vary with the various virtual flavor sensations perceived. The study presented two apparatuses to simulate real-world experiences: 1) A virtual beverage glass that mimiced the mocktail or cocktail glassware through which the liquid was visible. 2) A virtual coffee mug, which imitated a coffee mug or travel mug through which the liquid inside the cup was generally not visible through the cup.

### Participants

A sample set of 30 people were chosen for the study with both the apparatuses. Participants (also referred to as users) were above 18 years of age and did not have any color, taste, smell, and/or temperature blindness. They were instructed to refrain from consuming any food or beverages with heavy flavors such as coffee or spicy food an hour before the experiment. Furthermore, they were asked not to wear any cologne during the study and while attending their sessions to minimize external effects on the stimuli configurations. Participants gave consent before the study, and each session took a maximum of approximately 20 min.

### Stimuli Configurations

The study was conducted in an air-conditioned research laboratory with minimum interference. A quiet, odor-free environment was maintained to ensure a controlled testing environment. None of the people entering the room were allowed to wear any fragrance products during the study. The time gap between each participant was a minimum of 30 minutes to get rid of any remaining smells in the room. For the virtual beverage glass, there were 11 different stimuli configurations in four different categories, as described in Table 1 presented below.

TABLE 1
Stimuli combinations for virtual beverage glass.

| Modality | Conditions | | |
|---|---|---|---|
| Color | Red | Green | Yellow |
| Taste | Salt | Sour | |
| Smell | Lemon | Chocolate | Spirit |
| Combinations | Lemon + Sour + | Chocolate + | Spirit + Sour + |
| | Yellow | Salt + Red | Green |

Different stimuli configurations were selected to cover various combinations of visual, electric taste, smell, and thermal sensations. When choosing color stimuli, special attention was given to select natural colors from common food and beverages, in addition to the visibility of certain colors through plain water and glassware. For example, the blue color was not selected as it was less favored in food products, and the color blue was considered to be an atypical food color as there are very few naturally occurring blue colored foods. Saltiness and sourness were selected as they are the two prominent taste sensations reported via electrical stimulation on the tongue. Familiar smells such as lemon, chocolate, and spirit-like (alcohol) essential oils were used to formulate smell stimuli.

Similarly, for the virtual coffee mug, there were 11 different stimuli combinations under three different categories, as in Table 2 (shown below). Familiar smells such as coffee, chocolate, and mint were used with hot and cold thermal sensations in the virtual coffee mug. Hot and cold sensations were created by providing thermal stimuli that were 5° C. hotter or cooler from room temperature, respectively. Bottled-water that was taste and odor-free was utilized to maintain the uniformity of the base drink and to avoid any influence on the participants' flavor and emotion perceptions based on the water quality. The devices were sanitized using 70% isopropyl alcohol wipes before and after each participant's testing session.

TABLE 2
Stimuli combinations for virtual coffee mug.

| Modality | Conditions | | |
|---|---|---|---|
| Smell | Coffee | Chocolate | Mint |
| Temperature | Hot | Cold | |
| Combinations | Hot + Coffee | Hot + Chocolate | Hot + Mint |
| | Cold + Coffee | Cold + Chocolate | Cold + Mint |

### Experimental Method

Described herein are experimental methods used to conduct the experiments described herein and determine the relative amount of liking participants had of flavors.

For the virtual beverage glass, 30 participants (17 male and 13 females) in the age range of 18-37 years (M=26.42, S.D=4.8) were studied. For the virtual coffee mug, 29 participants (17 males and 12 females) in the age range of 18-40 years (M=22.89, S.D=5.23) were studied.

Each participant had 11 samples to test and responded to a series of questions following each sample. The participants took approximately 2 or 3 sips before they answered questions about each sample.

During the study with the virtual beverage glass, the participants were allowed to fill their apparatus with water as needed between the different samples. For the virtual coffee mug, one water-filled cup was sufficient for a participant. Participants were informed that they are drinking plain water using both apparatus as per the Institutional Review Board (IRB) approval.

The presentation order of different samples was counterbalanced to avoid the order bias. After each sample, participants were asked to complete a questionnaire to record perceived flavor sensations (in terms of four primary taste sensations), liking towards the stimuli (overall liking, smell liking, taste liking, color liking, and temperature liking), and emotions mediated through the stimuli. After each stimulus, flavor sensations were recorded in terms of salty, sour, sweet, and bitter sensations on the JAR (Just about right) scale. The JAR Scale is commonly used to evaluate the appropriateness of the level of a particular attribute in a product on a five-point categorical scale range from ‘much too little’ to ‘much too strong’. For example, the JAR scale helped to determine whether a specific taste sensation is too high, too low, or just about right in a particular virtual flavor configuration.

Participants also rated the liking towards each stimulus on a 9-point hedonic scale, which ranges from ‘extremely dislike’ to ‘extremely like’. Moreover, for each stimulus, the participants were asked to check all emotions that they can relate to on a check-all-that-apply (CATA) scale. The CATA approach was considered to be a quick and straightforward method to gather participants' feedback. The approach required little cognitive effort as they selected all emotions that apply related to a particular stimulus. Thus, the CATA scale was incorporated to evaluate participants' emotions, and the list of emotions was provided based on the EsSence Profile.

### Experimental Results

The main findings from the user experiment with two apparatuses (virtual beverage glass and virtual coffee mug) are discussed herein. Participants' responses concerning different flavor stimuli and mediated emotions are recorded in a similar manner for both apparatuses. While the virtual beverage glass provided feedback on the overall liking, smell liking, taste liking, and color liking, the virtual coffee mug provided feedback on overall liking, smell liking, taste liking and temperature liking based on different modalities included in each apparatus. A one way ANOVA was conducted to determine significant differences based on p-values≤0.05. Applicants conducted follow up post hoc analysis where necessary.

### Perceptions of Basic Taste Sensations with Different Flavor Stimuli

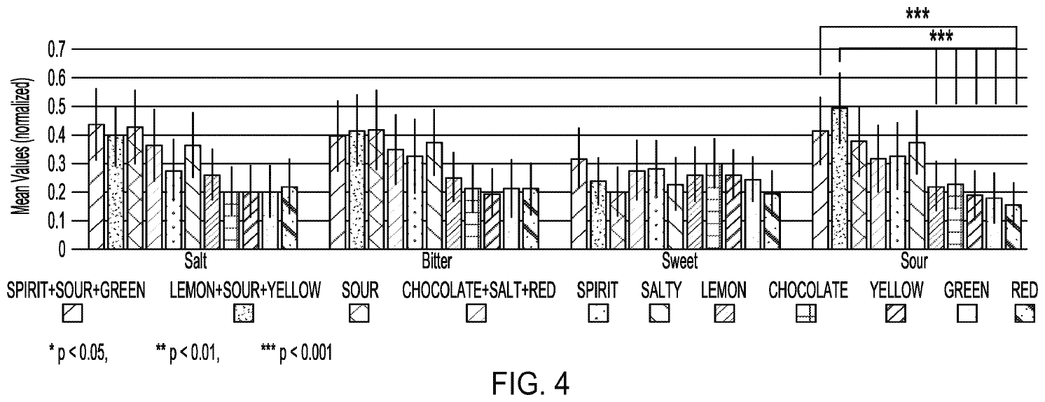

Perception of different flavor stimuli was evaluated using both apparatuses in terms of four primary taste sensations: salty, sour, sweet, and bitter. The strength of stimuli was ranked by participants on a 5 point “JAR (just about right) scale”. As shown in FIG. 4, results from the virtual beverage glass show that some stimuli were perceived to be in the range of ‘too weak’ to ‘just about right’. There were no significant differences recorded in the perception of salt, sour, and sweet sensations. In salty and bitter sensations, there was a clear trend from color-only stimuli (perceived as too weak) to combined stimuli (perceived as just about right). In general, sweet sensations were perceived as being ‘too weak’ during almost all the stimuli configurations tested without any significant differences. For sour sensations, a significant difference in perception was found using one way ANOVA, F(10, 329)=4.452, p≤0.0001. Post hoc tests with Tukey's HSD and Bonferroni pairwise comparisons revealed that sour perception is significantly higher in 1) Lemon+Sour+Yellow stimuli (M=0.492) compared to Lemon smell only (M=0.218, p=0.011), Chocolate smell only (M=0.226, p=0.016), Yellow color only (M=0.185, p=0.002), Green color only (M=0.177, p=0.001), and Red color only (M=0.157, p=0.00) stimuli, and 2) Spirit+Sour+Green stimuli (M=0.411) compared to Red color-only (M=0.153, p=0.023) stimulus.

There is a slight trend towards increased perception when multiple modalities are combined. Noticeably, when cold temperature+coffee smell was combined, there was an increased perception of the flavor. In both the virtual coffee mug and virtual beverage glass, combining multiple modalities enhanced the flavor perception. In addition, increasing intensity levels of either smell and/or color stimuli resulted in individual having more intense perceptions of flavors. This result was a particularly surprising finding. This finding is significant as it demonstrates that a combination of stimuli and modules that were not specifically targeted to the tongue were still able to alter flavor perception to a significant and unexpected degree.

### Participants' Likeness Towards Specific Virtual Flavor Stimuli

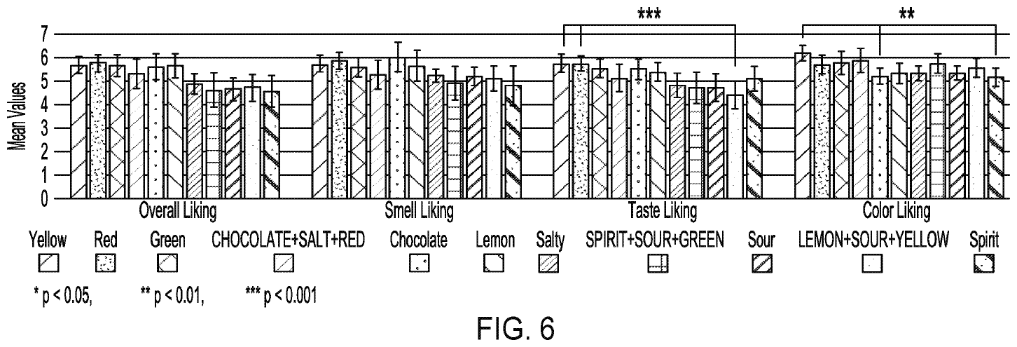

Participants' likeness scores towards different virtual flavor stimuli delivered through the virtual beverage glass are presented in FIG. 6. There were no significant differences reported in both overall liking and smell liking categories.

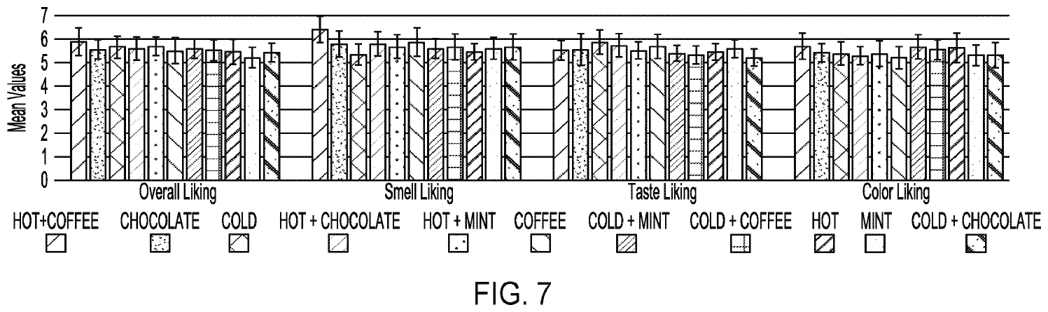

Interestingly in both categories, stimuli configured using color only (Yellow, Green, and Red) as well as Chocolate and Lemon smells showed an increasing trend in receiving higher liking scores when compared to other stimuli. Notably, a significant difference in liking scores was found in the taste liking category using one way ANOVA, F(10, 329)=3.131, p=0.001. Post hoc tests with Tukey's HSD and Bonferroni pairwise comparisons revealed that 1) Yellow only (M=5.742, p=0.013) and Red only (M=5.742, p=0.013) conditions received significantly higher liking scores compared to Lemon+Sour+Yellow stimuli (M=4.419). In Color liking, a significant difference in liking scores was found using one way ANOVA, F(10,329)=2.510, p=0.006. Post hoc tests with Tukey's HSD and Bonferroni pairwise comparisons revealed that 1) Yellow only (M=6.194) condition received significantly higher liking scores compared to Chocolate smell only stimulus (M=5.194, p=0.025) and Spirit smell only stimulus (M=5.161, p=0.017). The post hoc analysis conducted on the participants' liking scores recorded using a virtual coffee mug showed a trend in the liking towards different stimuli, as shown in FIG. 7. Though there was no significant difference in the overall liking, smell liking, taste liking, and temperature liking concerning the different stimuli presented, the analysis clearly showed that the liking scores were in the higher range. The high scores denoted that the participants, overall, liked the stimuli configurations. Notably, none of the stimuli liking scores were found to be in the dislike range. These results demonstrate that participants, in general, liked the stimuli configurations, the exemplary apparatuses, and the approaches.

### Participants' Likeness Towards Specific Virtual Flavor Stimuli

Stimulus combinations for the virtual beverage were combined into four different groups. The groups were as follows, color (containing the red, green, and blue color stimuli), electric taste (containing the salty and sour taste stimuli), smells (lemon, chocolate, and spirit smell stimuli), and combinations (containing yellow+sour+lemon, chocolate+salted+red, and spirit+sour+green). These groups were chosen because the groups shared similar kinds and number of stimuli. For example, in the color grouping, the virtual beverage only delivered color stimuli from a color module, no taste stimulus (e.g., using an electric taste module) and no smell (e.g., using a smell module) stimulus were provided to users. In the following section, differences within and between groupings are addressed.

Differences in overall liking, smell liking, taste liking, and temperature liking were compared within different groupings of stimuli. Repeated measures ANOVAs were conducted to test if there were differences in liking within groups. This was done to determine if a particular stimuli (e.g., a particular color) or a particular combination of stimuli within each group were not producing drastically different results so that intensity and liking scores could be collapsed. There were no differences within the groups for both smell liking and taste liking (p's<0.05). For overall liking, there was no difference within the color, electric taste, and combination group (p's<0.05). There was a significant difference in overall liking within the smell group (F(2, 60)=5.41, p=0.007, η²=0.091). Tukey's post-hoc analyses revealed that the spirit smell is overall liked less than the lemon smell (p=0.015) and the chocolate smell (p=0.018).

Similarly, differences in perception of the sensations of salty, sour, sweet, and bitter were compared within different groupings of the stimulus combinations. Repeated measures ANOVAs were conducted to test if there were differences in perception of sensations within the groups. There were no differences within groups for the perception of salt, sweet, or bitter. For the perception of sour, there were significant differences within the smell group F(2, 60)=4.76, p=0.0012, η²=0.03, and the combination group F(2, 60)=4.44, p=0.016, η²=0.049. Within the smell group, the spirit smell elicited a stronger sensation of sour as compared to both the lemon (p=0.02) and the chocolate (p=0.034) smell. In the combination group, the lemon+sour+yellow combination was perceived as being more sour compared to the chocolate+salted+red combination (p=0.012).

### Comparison of Virtual Beverage Groups in Terms of Taste

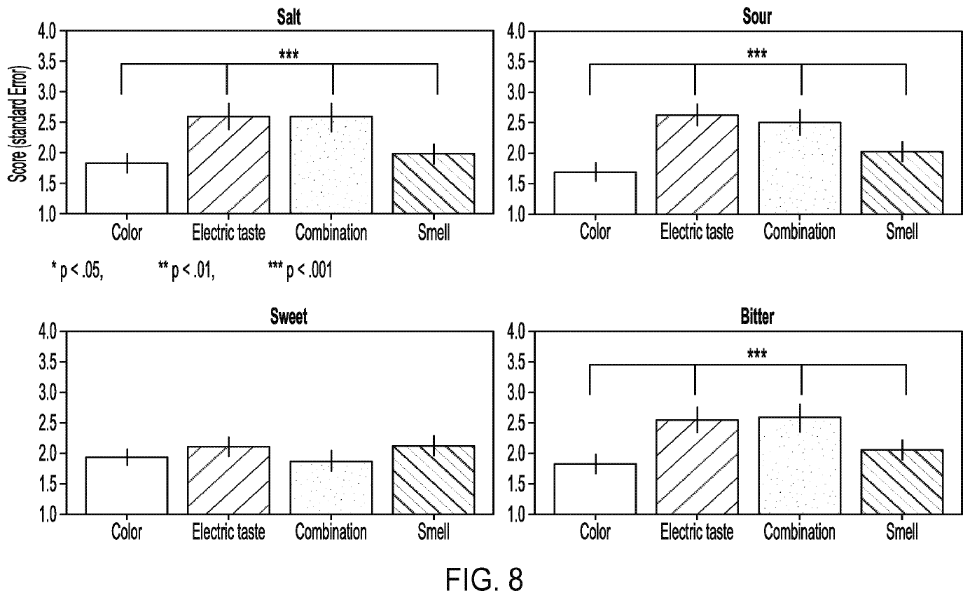

Here, the perception of different virtual flavor stimuli groupings (color, electric taste, smell, and combination) were evaluated in a virtual beverage in terms of four primary taste sensations: salty, sour, sweet, and bitter. Initial inspection of data reveals differences for salty, sour, and bitter sensations (see, e.g., FIG. 8). FIG. 8 is a series of graphs showing average intensity scores of different stimuli combinations in terms of primary taste sensations reported from the virtual beverage glass. A repeated measure ANOVA revealed a significant difference for perception of salty, F(3, 90)=8.96, p<0.001, =0.092. Tukey's post-hoc comparisons reveal that the electric taste and the combination groups are not different from each other (p>0.05) and are both greater than the color and smell groups (p<0.05). Significant differences in the perception of sour were found, F(3, 90)=14.3, p<0.001, η²=0.12. Similar to the perception of salty, the electric and the combination groups were higher in sour than the color and smell groups (p<0.05). There were also significant differences found in the perception of bitter F(3, 90)=9.94, p=<0.001, η²=0.077. The perception of bitter was greater in the electric taste group and the combination group as compared to the color and smell group p<0.05). There was no statistically significant difference between the groups for the perception of sweet. Overall, the electric taste group and the combination group showed higher flavor sensations.

### Comparing the Virtual Beverage Groups in Terms of Liking

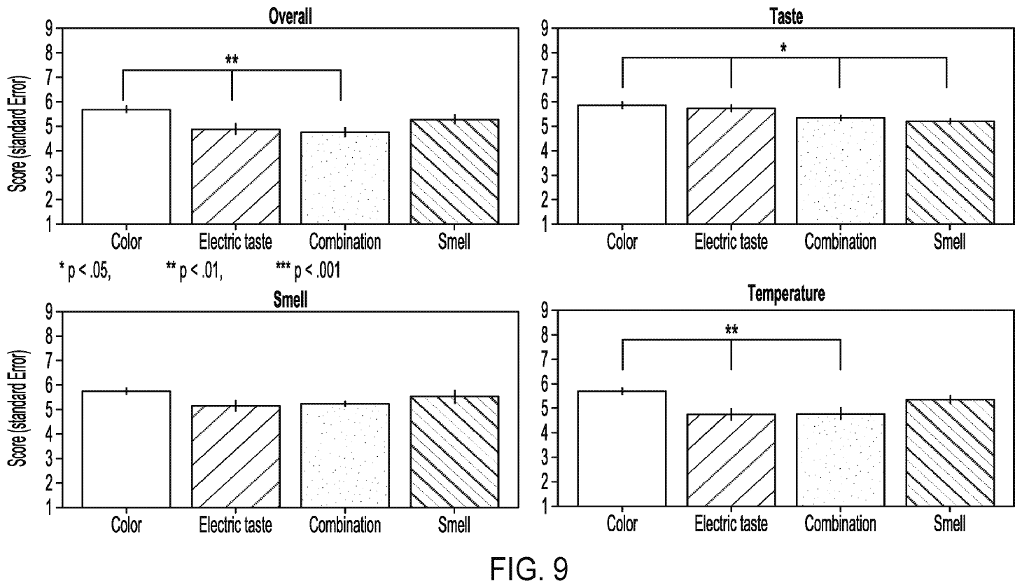

FIG. 9 shows a comparison of participants' overall liking, smell liking, taste liking, and temperature liking of different stimuli groupings. The groupings are compared herein to understand how the different types of stimuli configurations affect the liking of a virtual beverage.

Initial inspection of the data suggested that there was a general liking for the color group as compared to the combination group (FIG. 9). A repeated measures ANOVA showed a significant effect of stimulus group on overall liking (F(3, 90)=5.82, p<0.001, η2=0.10), taste liking (F(3, 90)=8.00, p<0.001, η²=0.12), and temperature liking (F(3, 90)=5.69, p<0.001, η²=0.11). Tukey's post-hoc analyses showed that participants overall liked the color group more than both the electric taste group (p=0.002) and the combination group (p=0.008). Participants liked the taste of the color and the combination groups more than both the electric taste and the smell groups (p's<0.05). Finally, participants liked the temperature of the color group more than the electric taste (p=0.006) and the combination group (p=0.004). There was no difference in how the participants liked the smell of the groups (p=0.055). Overall, participants liked the color group the most but also enjoyed the taste of the combination group.

### Grouping Virtual Coffee Mug Modalities

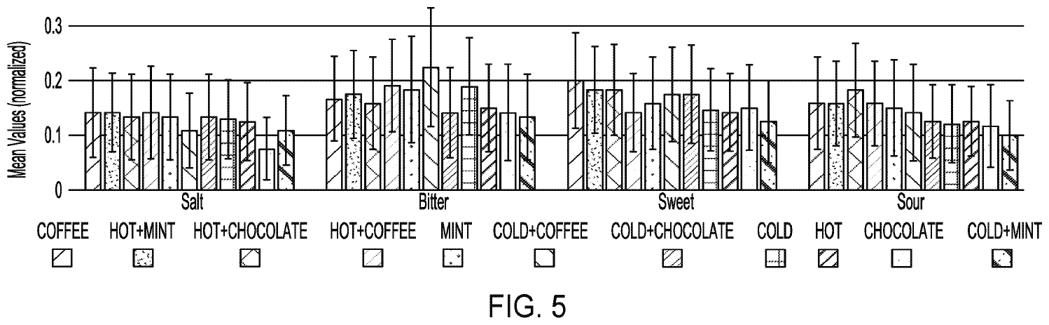

In the same way that there were groupings created a priori for the virtual beverage, groupings for stimuli delivered by a virtual coffee device as described herein were created. They were as follows: flavor (chocolate, mint, and coffee flavorings only), hot flavor (hot temperate+chocolate, hot temperature+mint, and hot temperature+mint), cold flavor (cold temperate+chocolate, cold temperature+mint, and cold temperature+mint), hot temperature, cold temperature, and no stimulation. The groups, flavor, hot flavor, and cold flavor were examined for within group differences in terms of the primary sensations (salty, sour, sweet, and bitter) (e.g., FIG. 5) and liking scores (overall, taste, smell, and temperature) (e.g., FIG. 7). FIG. 5 is graph of average intensity scores of different stimuli from a virtual coffee device. Initial inspection of the data suggest that overall there are few, if any differences within groupings. Repeated measures ANOVAs confirmed that there were few differences within the groupings. Within the groups, there was no difference in terms of the primary sensations (p>0.05) and for the liking scores there was only one difference. There was a significant effect of smell liking within the hot flavor group, F(2, 54)=4.06, p=0.023, η²=0.062. The hot+coffee stimuli combination had greater smell liking than the hot+mint (p=0.022) stimuli combination. Other than users liking the smell of the coffee when paired with a hot temperature more than the smell of the mint, there were no observed differences within the groups.

### Emotional Responses and Associations with Virtual Flavor Stimuli

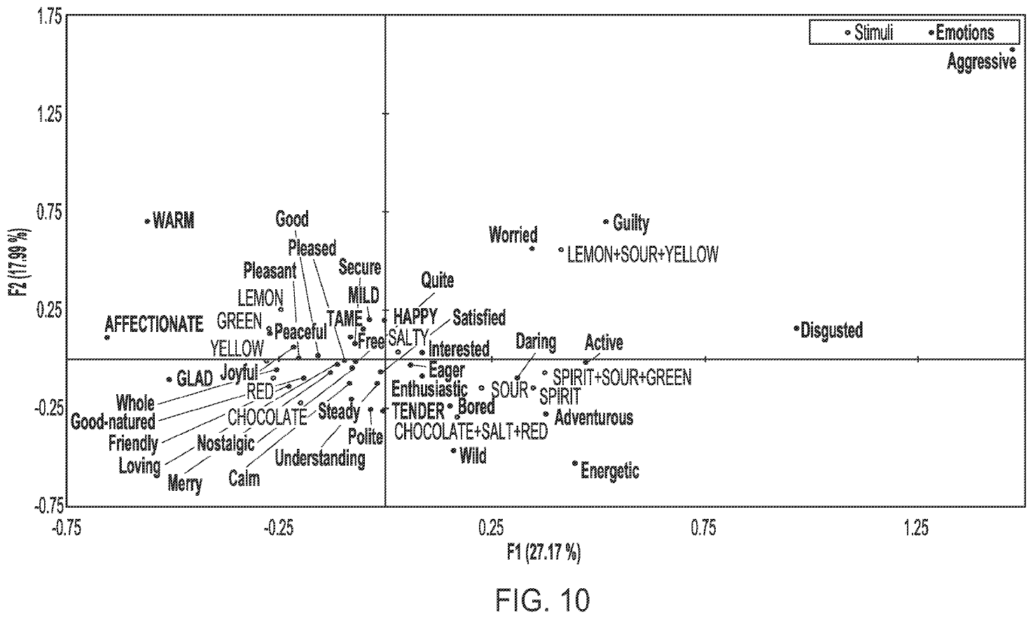

FIG. 10 depicts the various emotions mediated by different virtual flavor stimuli delivered via the virtual beverage glass on a correspondence analysis factor map. The correspondence analysis factor map was created through the use of principle component analysis (PCA). Analysis of the data using principle components reduced redundancy in the data. Principal components (PCs) account for most of the variance in the observed variables so that the more variance in the factors that are presented (e.g., F1 and F2 herein), then more variability is accounted for in the graph that is presented. The axes revealed 45.16% of the variance in the data. The F1 axis accounts for positive (affectionate) and negative (disgusted) emotions while the F2 axis illustrates the emotions related to energy and extends between the positive (energetic) and negative (aggressive) emotions. Emotions elicited from color stimuli were related to comforting emotions such as ‘peaceful’, ‘pleasant’, and ‘friendly’. Among the electric taste sensations, salty was closely related to ‘interested’ and ‘eager’ emotions. The sour taste was not particularly related to a specific emotion; however, it was on the lines of a ‘boring’ emotion. The smell of Chocolate was closely associated with a lot of positive emotions like ‘friendly’, ‘calm’, ‘polite’, ‘steady’, and ‘understanding’. The Lemon smell did not have a close association with any specific emotion. The participants associated the spirit smell with a ‘daring’ emotion, which was interesting. Following a similar trend, participants reported different emotions when combined stimuli of color, taste, and smell were provided. For example, the Spirit smell was associated with ‘daring’ emotion. When the spirit smell was combined with the Sour taste and Green color, the participants felt ‘active’. On the contrary, the combined stimuli of Lemon smell, Sour taste, and Yellow color, which was intended to imitate a lemonade-like experience was related to ‘guilty’ and ‘worried’ emotions.

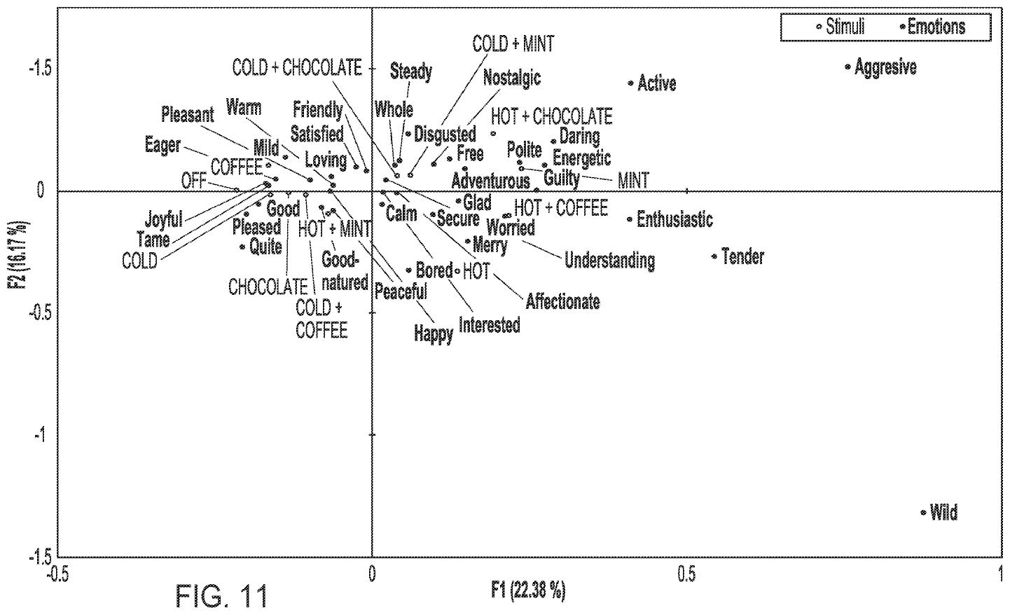

FIG. 11 depicts emotions elicited by different virtual flavor stimuli delivered via a virtual coffee mug on the correspondence analysis factor map. The axes show a 38.54% of the variability in the data. The F1 axis displays the emotions related to ‘disgusted’ and ‘bored’, while the F2 axis extends between positive (“good”) and negative (“guilty”) emotions.

As explained herein, there was a diverse range of emotions elicited when delivering virtual flavor sensations from either the virtual coffee or the virtual beverage glass. The emotions elicited from a virtual coffee mug were closely attached to different stimuli when compared to emotions elicited using a virtual beverage glass.

It is interesting to notice that the hot stimulus was not enjoyed when delivered to the participants alone as it was related to ‘bored’ emotion. However, when the hot stimulus was combined with smells, the participants' responses were significantly altered. For example, hot+chocolate stimuli were associated with ‘daring’ emotion, while hot+coffee was related to ‘worried’ emotion. In contrast, hot+mint stimuli were associated with ‘peaceful’ emotion.

The cold stimulus was associated with several emotion clusters when simulated alone or in combinations. The cold stimulus was related to ‘good’ and ‘pleased’ emotions while cold+coffee was related to ‘good’, ‘good-natured’, ‘peaceful’ emotions. Similarly, cold+chocolate stimuli were perceived to be rich in ‘understanding’, ‘interesting’ and ‘friendly’ emotions, and cold+mint stimuli were closely associated with ‘whole’, ‘nostalgic’, ‘steady’, and ‘free’ emotions. The mint smell only stimulus was associated with ‘polite’ and ‘adventurous’ emotions. A similar trend was observed with coffee and chocolate smell stimuli, which were associated with ‘mild’, ‘loving’, ‘joyful’, ‘pleasant’, and ‘warm’ emotions. Almost all the stimuli given in the virtual coffee mug were related to positive emotions, which ultimately represents a good user experience with not only the apparatus but also virtual flavor sensations.

### Discussion of Experimental Results

The results of the experiments discussed herein revealed that participants 1) perceived various flavor sensations when presented with different stimuli configurations, 2) generally liked different simulated flavor sensations, 3) distinguished different basic taste sensations as per the stimuli presented, and 4) associated different emotions with various simulated flavor sensations.

One significant finding that was unexpected was that the participants disliked none of the stimuli configurations. This finding is encouraging as the way virtual flavor components were delivered contrasts significantly from a traditional drinking experience. In a traditional experience where an individual is consuming food, volatile aromatic compounds are released retronasally through mastication inside the mouth. In an approach described herein, users were stimulated by: smell stimulus released orthonasally; an electric taste stimulus applied to the tip of the tongue; and thermal stimulus applied to the outer lips. Approaches discussed herein for delivering flavor sensations were first time experiences for all participants. This might have had an influence on their liking scores through the “Neophic effect”. This effect results in a fear of trying new things, even in a controlled experimental environment. Furthermore, the two apparatuses developed were a little heavier and different from everyday utensils; thus, it might also have negatively affected participants' responses.

In addition, stimuli configurations used for this example were not calibrated towards individual preferences. The flavor combinations delivered to users through a flavor profile may be further calibrated based on individual preferences. The preferred intensity levels of colors, smells, electric stimuli, and thermal stimuli can be varied based on different participants. The responses received for sweet taste sensation were determined to be weak based on stimuli delivered from the virtual beverage glass.

The combined (color+taste+smell) stimuli was found to deliver a wholesome, ‘just about right’ experience. For instance, in sour taste perception, Lemon+Sour+Yellow reports higher sour intensity levels compared to electric sour sensation delivered alone.

The virtual coffee cup was designed to only provide smell and thermal stimuli, but not any taste stimulus (e.g., via an electrical stimulus to the tongue). However it was surprising that in the absence of an electric taste stimulus that users reported sensing flavors as discussed herein despite drinking only plain water based on the combination of heat and smell stimuli alone. Accordingly, in certain embodiments, combinations of modules and/or stimuli as disclosed herein may be provided to a user.

Elicited emotions from different virtual flavor sensations were generally considered to be positive emotions. It was also observed that the emotions reported from the virtual beverage glass were recognized as calm, peaceful, and pleasant emotions while from the virtual coffee mug were identified as energetic, happy, and merry. These emotions are generally also associated with soft drinks and coffee.

None of the stimuli were related to disgust or anger. These findings confirmed that participants enjoyed most of the stimuli configurations. Even more smell and color sensations are possible than those disclosed herein.

### Exemplary Virtual Beverage Device 1

In one aspect, the disclosure encompasses systems for delivering a simulated flavor sensation to a user, the system comprising one or more sensory modules, wherein the sensory modules comprise: a smell module for providing a scent corresponding to the simulated flavor sensation to the user and positioned to deliver the scent to the nose of the user; and a thermal module for providing a thermal stimulus to a portion of the tongue and/or a portion of the upper lip and/or the lower lip of the user and/or a portion of an area between the upper lip and the nose of the user and/or one or more nerves of the user.

In certain embodiments, the systems further comprise: an image generating device for generating one or more images; and a communication module for transmitting the simulated flavor sensation corresponding to at least one of the one or more images to the processor. In certain embodiments, the instructions, when executed by the processor, cause the processor to alter the data corresponding to the simulated flavor sensation based on the scene data.

In certain embodiments, the systems further comprise a structural frame adaptably configured to be mounted on the head of the user and wherein the image generating device is mounted to the structural frame for user observation of the video scenes.

In certain embodiments, the smell module comprises one or more smell containers, wherein each of the smell container comprises one or more compositions having a fragrance. In certain embodiments, the fragrance comprises an essential oil. In certain embodiments, the smell module further comprises one or more air-pumps and wherein each of the one or more smell containers is operably interfaced with at least one of the one or more air-pumps. In certain embodiments, the smell container comprises a medium to absorb and/or contain the one or more compositions.

In certain embodiments, the thermal module comprises a heating and/or cooling element. In certain embodiments, the heating and/or cooling element is a thermoelectric heating and/or cooling element. In certain embodiments, the cooling and/or heating element is operatively interfaced with a thermally conductive material, wherein the thermally conductive material is in communicative contact with the portion of the tongue and/or the portion of the upper lip and/or the lower lip of the user and/or the portion of an area between the upper lip and the nose of the user and/or the one or more nerves of the user. In certain embodiments, the cooling and/or heating element is operatively interfaced with a thermally conductive material, wherein the thermally conductive material is in communicative contact with the tongue of the user. In certain embodiments, the thermally conductive material is and/or comprises a metal. In certain embodiments, the thermally conductive material is 95% silver.

In certain embodiments, the thermal stimulus is from 15° C. to 40° C.

In certain embodiments, the thermal stimulus comprises rapidly heating and cooling from 20° C. to 38° C.

In certain embodiments, the systems comprise a vessel for containing a liquid.

In certain embodiments, at least one of the one or more sensory modules are mounted to a vessel.

In certain embodiments, the one or more sensory modules further comprises one or more additional modules, wherein the one or more additional modules stimulate different attributes of a flavor sensation.

In certain embodiments, the one or more nerves of the user comprises one or more cranial nerves. In certain embodiments, the one or more cranial nerves is or comprises a trigeminal nerve.

### Exemplary Virtual Beverage Device 2

In one aspect, the disclosure encompasses systems for delivering a simulated flavor sensation to a user, the system comprising one or more sensory modules wherein the sensory modules comprise: a smell module for providing a scent corresponding to the simulated flavor sensation to the user and positioned to deliver the scent to the nose of the user; an electric taste module for electrically stimulating the tongue of the user; and a color projection module for providing visual cues to the user.

In certain embodiments, the systems further comprise: a processor of a computing device; and a memory having instructions stored thereon, wherein the instructions, when executed by the processor, cause the processor to: receive and/or access data corresponding to the simulated flavor sensation; and use the data corresponding to the simulated flavor sensation to deliver the simulated flavor sensation to the user using at least one of the one or more sensory modules. In certain embodiments, the systems comprise at least one electric taste module and the instructions cause the processor to deliver an electric stimulus corresponding to the simulated flavor sensation using the electric taste module to the tongue of the user. In certain embodiments, the electric stimulus comprises a current from 0 to 200 μA. In certain embodiments, the electric stimulus comprises a frequency from 0 to 1200 Hz.

In certain embodiments, the electric taste module comprises: a set of electrodes; and a function generator operably connected to the set of electrodes. In certain embodiments, the set of electrodes comprises at least two electrodes and wherein the electrodes are spaced apart by approximately 2 mm. In certain embodiments, the set of electrodes comprises at least two electrodes and wherein the electrodes are spaced apart by 0.1 mm to 4 mm. In certain embodiments, the function generator is and/or comprises a constant current source. In certain embodiments, the set of electrodes is and/or comprises a conductive material. In certain embodiments, the conductive material is and/or comprises a metal or a metal alloy. In certain embodiments, the metal or metal alloy comprises at least 95% silver. In certain embodiments, the conductive material comprises a conductive polymer.

In certain embodiments, the smell module comprises one or more smell containers, wherein each of the smell container comprises one or more compositions having a fragrance. In certain embodiments, the fragrance comprises an essential oil. In certain embodiments, the smell module further comprises one or more air-pumps and wherein each of the one or more smell containers is operably interfaced with at least one of the one or more air-pumps. In certain embodiments, the smell container comprises a medium to absorb and/or contain the one or more compositions.

In certain embodiments, the color module comprises one or more illumination sources.

In certain embodiments, the color module comprises a diffusive element to disperse and/or soften the at least one of the one or more illumination sources.

In certain embodiments, the system comprises a vessel for containing a liquid.

In certain embodiments, at least one of the one or more sensory modules are mounted to a vessel.

In certain embodiments, the one or more sensory modules further comprises one or more additional modules, wherein the one or more additional modules stimulate different attributes of a flavor sensation.

### Computer System and Network Environment

As discussed herein, it is contemplated that various embodiments, may be included as a part of a networked environment. The description in this section is intended to provide exemplary configurations and parameters for use with some embodiments, and it is not meant to be limiting.

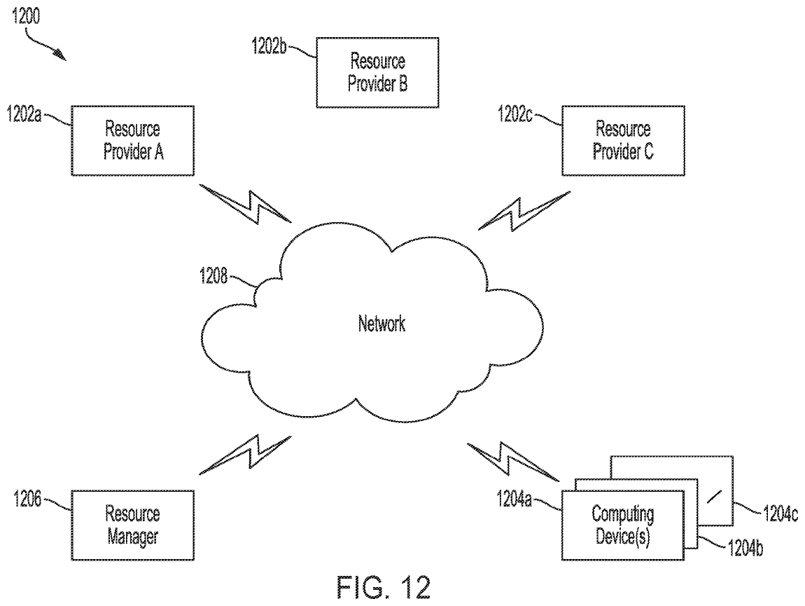

As shown in FIG. 12, an implementation of a network environment 1200 for use in providing systems, methods, and architectures for delivering a simulated flavor sensation to a user as described herein is shown and described. In brief overview, referring now to FIG. 12, a block diagram of an exemplary cloud computing environment 1200 is shown and described. The cloud computing environment 1200 may include one or more resource providers 1202a, 1202b, 1202c (collectively, 1202). Each resource provider 1202 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1202 may be connected to any other resource provider 1202 in the cloud computing environment 1200. In some implementations, the resource providers 1202 may be connected over a computer network 1208. Each resource provider 1202 may be connected to one or more computing device 1204a, 1204b, 1204c (collectively, 1204), over the computer network 1208. In certain embodiments, one or more computing devices may be a part of devices, methods, and/or system(s) disclosed herein. For example, a resource provider may store flavor profiles and/or computer applications to create and/or alter flavor profiles.

The cloud computing environment 1200 may include a resource manager 1206. The resource manager 1206 may be connected to the resource providers 1202 and the computing devices 1204 over the computer network 1208. In some implementations, the resource manager 1206 may facilitate the provision of computing resources by one or more resource providers 1202 to one or more computing devices 1204. The resource manager 1206 may receive a request for a computing resource (e.g., a simulated flavor sensation, a flavor profile) from a particular computing device 1204. The resource manager 1206 may identify one or more resource providers 1202 capable of providing the computing resource requested by the computing device 1204. The resource manager 1206 may select a resource provider 1202 to provide the computing resource. The resource manager 1206 may facilitate a connection between the resource provider 1202 and a particular computing device 1204. In some implementations, the resource manager 1206 may establish a connection between a particular resource provider 1202 and a particular computing device 1204. In some implementations, the resource manager 1206 may redirect a particular computing device 1204 to a particular resource provider 1202 with the requested computing resource.

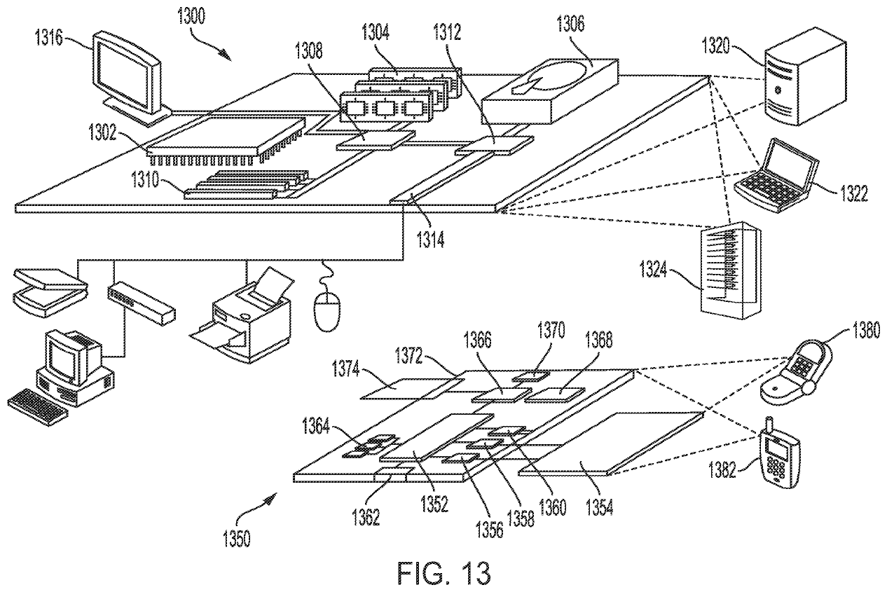

FIG. 13 shows an example of a computing device 1300 and a mobile computing device 1350 that can be used to implement the methods and systems described in this disclosure. The computing device 1300 is intended to represent various forms of digital computers, such as laptops, desktops, workstations, personal digital assistants, servers, blade servers, mainframes, and other application appropriate computers. The mobile computing device 1350 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1300 includes a processor 1302, a memory 1304, a storage device 1306, a high-speed interface 1308 connecting to the memory 1304 and multiple high-speed expansion ports 1310, and a low-speed interface 1312 connecting to a low-speed expansion port 1314 and the storage device 1306. Each of the processor 1302, the memory 1304, the storage device 1306, the high-speed interface 1308, the high-speed expansion ports 1310, and the low-speed interface 1312, are interconnected using various busses, and may be mounted on a common motherboard or in other manners as appropriate. The processor 1302 can process instructions for execution within the computing device 1300, including instructions stored in the memory 1304 or on the storage device 1306 to display graphical information for a GUI on an external input/output device, such as a display 1316 coupled to the high-speed interface 1308. In other implementations, multiple processors and/or multiple busses may be used, as appropriate, along with multiple memories and types of memory. Also, multiple computing devices may be connected, with each device providing portions of the necessary operations (e.g., as a server bank, a group of blade servers, or a multi-processor system). For example, as disclosed herein, a computing device may individually control a sensory module. In certain embodiments, each module may be operated independently through an independent and/or networked computing device. In certain embodiments, a single computing device may control multiple modules. Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1304 stores information within the computing device 1300. In some implementations, the memory 1304 is a volatile memory unit or units. In some implementations, the memory 1304 is a non-volatile memory unit or units. The memory 1304 may also be another form of computer-readable medium, such as a magnetic or optical disk.

The storage device 1306 is capable of providing mass storage for the computing device 1300. In some implementations, the storage device 1306 may be or contain a computer-readable medium, such as a floppy disk device, a hard disk device, an optical disk device, or a tape device, a flash memory or other similar solid state memory device, or an array of devices, including devices in a storage area network or other configurations. In certain embodiments, the computer-readable medium may store one or more flavor profiles and/or user preferences for flavor. Instructions can be stored in an information carrier. The instructions, when executed by one or more processing devices (for example, processor 1302), perform one or more methods, such as those described herein. For example, in certain embodiments, the instructions may provide a simulated flavor profile to a user through receiving a target flavor profile and stimulating a user based on the profile. The instructions can also be stored by one or more storage devices such as computer- or machine-readable mediums (for example, the memory 1304, the storage device 1306, or memory on the processor 1302).

The high-speed interface 1308 manages bandwidth-intensive operations for the computing device 1300, while the low-speed interface 1312 manages lower bandwidth-intensive operations. Such allocation of functions is an example only. In some implementations, the high-speed interface 1308 is coupled to the memory 1304, the display 1316 (e.g., through a graphics processor or accelerator), and to the high-speed expansion ports 1310, which may accept various expansion cards (not shown). In the implementation, the low-speed interface 1312 is coupled to the storage device 1306 and the low-speed expansion port 1314. The low-speed expansion port 1314, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1300 may be implemented in a number of different forms, as shown in the figure. For example, it may be implemented as a standard server 1320, or multiple times in a group of such servers. In addition, it may be implemented in a personal computer such as a laptop computer 1322. It may also be implemented as part of a rack server system 1324. Alternatively, components from the computing device 1300 may be combined with other components in a mobile device (not shown), such as a mobile computing device 1350. Each of such devices may contain one or more of computing devices 1300 and mobile computing devices 1350, and an entire system may be made up of multiple computing devices communicating with each other. For example, in certain embodiments a laptop or server may be used to deliver simulated flavor sensations simultaneously or individually to users.

The mobile computing device 1350 includes a processor 1352, a memory 1364, an input/output device such as a display 1354, a communication interface 1366, and a transceiver 1368, among other components. The mobile computing device 1350 may also be provided with a storage device, such as a micro-drive or other device, to provide additional storage. Each of the processor 1352, the memory 1364, the display 1354, the communication interface 1366, and the transceiver 1368, are interconnected using various buses, and several of the components may be mounted on a common motherboard or in other manners as appropriate.

The processor 1352 can execute instructions within the mobile computing device 1350, including instructions stored in the memory 1364. The processor 1352 may be implemented as a chipset of chips that include separate and multiple analog and digital processors. The processor 1352 may provide, for example, for coordination of the other components of the mobile computing device 1350, such as control of user interfaces, applications run by the mobile computing device 1350, and wireless communication by the mobile computing device 1350. In certain embodiments, the processor may provide, for example, for coordination of one or more sensory modules as disclosed herein.

The processor 1352 may communicate with a user through a control interface 1358 and a display interface 1356 coupled to the display 1354. The display 1354 may be, for example, a TFT (Thin-Film-Transistor Liquid Crystal Display) display or an OLED (Organic Light Emitting Diode) display, or other appropriate display technology. The display interface 1356 may comprise appropriate circuitry for driving the display 1354 to present graphical and other information to a user. In certain embodiments, graphical information may include information on a current and/or desired flavor profile. In certain other embodiments, graphical information may include information on user specific flavor profiles. The control interface 1358 may receive commands from a user and convert them for submission to the processor 1352. In addition, an external interface 1362 may provide communication with the processor 1352, so as to enable near area communication of the mobile computing device 1350 with other devices. The external interface 1362 may provide, for example, for wired communication in some implementations, or for wireless communication in other implementations, and multiple interfaces may also be used.

The memory 1364 stores information within the mobile computing device 1350. The memory 1364 can be implemented as one or more of a computer-readable medium or media, a volatile memory unit or units, or a non-volatile memory unit or units. An expansion memory 1374 may also be provided and connected to the mobile computing device 1350 through an expansion interface 1372, which may include, for example, a SIMM (Single In Line Memory Module) card interface. The expansion memory 1374 may provide extra storage space for the mobile computing device 1350, or may also store applications or other information for the mobile computing device 1350. Specifically, the expansion memory 1374 may include instructions to carry out or supplement the processes described above, and may include secure information also. For example, in certain embodiments the expansion memory may provide for instructions regarding the simulated flavor sensation, such that switching, adding, and/or removing the expansion memory alters the flavor profile. In this way, the expansion memory may act similarly to a gaming cartridge. In certain embodiments, the expansion memory 1374 may be provided as a security module for the mobile computing device 1350, and may be programmed with instructions that permit secure use of the mobile computing device 1350. In certain embodiments, the instructions provided may provide for user safety through the use of one or more safety measures as described herein. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1352), perform one or more methods, such as those described above. The instructions can also be stored by one or more storage devices, such as one or more computer- or machine-readable mediums (for example, the memory 1364, the expansion memory 1374, or memory on the processor 1352). In some implementations, the instructions can be received in a propagated signal, for example, over the transceiver 1368 or the external interface 1362.

The mobile computing device 1350 may communicate wirelessly through the communication interface 1366, which may include digital signal processing circuitry where necessary. The communication interface 1366 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1368 using a radio-frequency. In addition, short-range communication may occur, such as using a Bluetooth®, Wi-Fi™, or other such transceiver (not shown). In addition, a GPS (Global Positioning System) receiver module 1370 may provide additional navigation- and location-related wireless data to the mobile computing device 1350, which may be used as appropriate by applications running on the mobile computing device 1350.

The mobile computing device 1350 may also communicate audibly using an audio codec 1360, which may receive spoken information from a user and convert it to usable digital information. The audio codec 1360 may likewise generate audible sound for a user, such as through a speaker, e.g., in a handset of the mobile computing device 1350. Such sound may include sound from voice telephone calls, may include recorded sound (e.g., voice messages, music files, etc.) and may also include sound generated by applications operating on the mobile computing device 1350.

The mobile computing device 1350 may be implemented in a number of different forms, as shown in the figure. For example, it may be implemented as a cellular telephone 1380. It may also be implemented as part of a smart-phone 1382, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, systems and methods described herein can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input. In certain embodiments, systems and methods described herein, the computer may be incorporated with virtual reality and/or augmented reality systems as describe herein. For example, a display device may be mounted to the head of a user. In certain embodiments, the virtual reality and/or augmented reality system may also and/or alternatively incorporate audio cues to alter flavors based on sounds provided to the user, as described herein.

The systems and techniques described here can be implemented in a computing system that includes a back end component (e.g., as a data server), or that includes a middleware component (e.g., an application server), or that includes a front end component (e.g., a client computer or a cellphone having a graphical user interface or a Web browser through which a user can interact with an implementation of the systems and methods described herein), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other. For example, in certain embodiments, a cellphone may act as a client which received flavor profiles and/or simulated flavor sensations from a remote server.

In some implementations, the modules (e.g. data aggregation module 1330, mapping module 1350, specifications module 1370) described herein can be separated, combined or incorporated into single or combined modules. The modules depicted in the figures are not intended to limit the systems described herein to the software systems and architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

What is claimed is:

1. A system for delivering a simulated flavor sensation to a user, the system comprising one or more sensory modules, wherein the sensory modules are selected from the group comprising:
a smell module for providing a scent corresponding to the simulated flavor sensation to the user and positioned to deliver the scent to the nose of the user;
a thermal module for providing a thermal stimulus to a portion of the tongue and/or a portion of the upper lip and/or the lower lip of the user and/or a portion of an area between the upper lip and the nose of the user and/or one or more nerves of the user;
an electric taste module for electrically stimulating the tongue of the user; and/or
a color projection module for providing visual cues to the user.

2. The system of claim 1, wherein the system further comprises:
a processor of a computing device;
and a memory having instructions stored thereon, wherein the instructions, when executed by the processor, cause the processor to:
receive and/or access data corresponding to the simulated flavor sensation; and
use the data corresponding to the simulated flavor sensation to deliver the simulated flavor sensation to the user using at least one of the one or more sensory modules.

3. The system of claim 2, wherein the system comprises at least one electric taste module and the instructions cause the processor to deliver an electric stimulus corresponding to the simulated flavor sensation using the electric taste module to the tongue of the user.

4. The system of claim 3, wherein the electric stimulus comprises a current from 0 to 200 μA.

5. The system of claim 3 or 4, wherein the electric stimulus comprises a frequency from 0 to 1200 Hz.

6. The system of any one of the preceding claims, wherein the system further comprises:
an image generating device for generating one or more images; and
a communication module for transmitting the simulated flavor sensation corresponding to at least one of the one or more images to the processor.

7. The system of claim 6, wherein the instructions, when executed by the processor, cause the processor to alter the data corresponding to the simulated flavor sensation based on the scene data.

8. The system of claim 6 or 7, wherein the system further comprises a structural frame adaptably configured to be mounted on the head of the user and wherein the image generating device is mounted to the structural frame for user observation of the video scenes.

9. The system of any one of the preceding claims, wherein the electric taste module comprises:
a set of electrodes; and
a function generator operably connected to the set of electrodes.

10. The system of claim 9, wherein the set of electrodes comprises at least two electrodes and wherein the electrodes are spaced apart by approximately 2 mm.

11. The system of claim 9, wherein the set of electrodes comprises at least two electrodes and wherein the electrodes are spaced apart by 0.1 mm to 4 mm.

12. The system of any one of claims 9 to 11, wherein the function generator is and/or comprises a constant current source.

13. The system of any one of claims 9 to 12, wherein the set of electrodes is and/or comprises a conductive material.

14. The system of claim 13, wherein the conductive material is and/or comprises a metal or a metal alloy

15. The system of claim 14, wherein the metal or metal alloy comprises at least 95% silver.

16. The system of any one of claims 13 to 15, wherein the conductive material comprises a conductive polymer.

17. The system of any one of the preceding claims, wherein the smell module comprises one or more smell containers, wherein each of the smell container comprises one or more compositions having a fragrance.

18. The system of claim 17, wherein the fragrance comprises an essential oil.

19. The system of any of the preceding claims, wherein the smell module further comprises one or more air-pumps and wherein each of the one or more smell containers is operably interfaced with at least one of the one or more air-pumps.

20. The system of any one of claims 17 to 19, wherein the smell container comprises a medium to absorb and/or contain the one or more compositions.

21. The system of any one of the preceding claims, wherein the thermal module comprises a heating and/or cooling element.

22. The system of claim 21, wherein the heating and/or cooling element is a thermoelectric heating and/or cooling element.

23. The system of claim 21 or 22, wherein the cooling and/or heating element is operatively interfaced with a thermally conductive material, wherein the thermally conductive material is in communicative contact with the portion of the tongue and/or the portion of the upper lip and/or the lower lip of the user and/or the portion of an area between the upper lip and the nose of the user and/or the one or more nerves of the user.

24. The system of any one of claims 21 to 23, wherein the cooling and/or heating element is operatively interfaced with a thermally conductive material, wherein the thermally conductive material is in communicative contact with the tongue of the user.

25. The system of claim 23 or 24, wherein the thermally conductive material is and/or comprises a metal.

26. The system of claim 25, wherein the thermally conductive material is 95% silver.

27. The system of any one of the preceding claims, wherein the thermal stimulus is from 15° C. to 40° C.

28. The system of any one of the preceding claims, wherein the thermal stimulus comprises rapidly heating and cooling from 20° C. to 38° C.

29. The system of any one of the preceding claims, wherein the color module comprises one or more illumination sources.

30. The system of any one of the preceding claims, wherein the color module comprises a diffusive element to disperse and/or soften the at least one of the one or more illumination sources.

31. The system of any one of the preceding claims, wherein the system comprises a vessel for containing a liquid.

32. The system of any one of the preceding claims, wherein at least one of the one or more sensory modules are mounted to a vessel.

33. The system of any one of the preceding claims, wherein the system comprises an eating utensil for consuming a food item.

34. The system of any one of the preceding claims, wherein the one or more sensory modules further comprises one or more additional modules, wherein the one or more additional modules stimulate different attributes of a flavor sensation.

35. The system of any one of the preceding claims, wherein the one or more nerves of the user comprises one or more cranial nerves.

36. The system of claim 35, wherein the one or more cranial nerves is or comprises a trigeminal nerve.

37. A method of delivering a simulated flavor sensation to a user, the method comprising:
receiving a target flavor profile corresponding to a simulated flavor sensation; and
stimulating the user based on at least the target flavor profile by performing one or more of the following:
dispensing, to the nose of the subject, one or more fragrances corresponding to the target flavor profile;
heating or cooling a portion of the tongue of the user and/or a portion of an upper lip of the user and/or a portion of an area between the upper lip and nose and/or one or more nerves using a thermal module, wherein a temperature of the thermal module corresponds to the target flavor profile;
electrically stimulating the tongue of the user corresponding to the target flavor profile; and/or
projecting one or more colors of light corresponding to the target flavor profile.

38. The method of claim 37, wherein the step of stimulating the user further comprises stimulating the user based on one or more images shown to the user.

39. The method of claim 37 or 38, wherein electrically stimulating the tongue of the user comprises delivering an electrical signal to the tongue of the user.

40. The method of claim 39, wherein the electrical signal comprises a current from 0 to 200 μA.

41. The system of claim 37 or 40, wherein the electrical signal comprises a frequency from 0 to 1200 Hz.

42. The system of any one of claims 39 to 41, wherein the electrical signal corresponds to a bitter taste.

43. The system of any one of claims 39 to 42, wherein the electrical signal corresponds to a sour taste.

44. The system of any one of claims 39 to 43, wherein the electrical signal corresponds to a salty taste.

45. The method of any one of claims 37 to 44, wherein dispensing the one or more fragrances comprises blowing at least one of the one or more fragrances to the nose of the user.

46. The method of any one of claims 37 to 45, wherein dispensing the one or more fragrances further comprises pacing the dispensing of the one or more fragrances.

47. The method of any one of claims 37 to 46, wherein the heating or cooling comprises heating or cooling a heating and/or cooling element to a temperature from 15° C. to 40° C.

48. The method of any one of claims 37 to 47, wherein the heating or cooling corresponds to a sweet taste and comprises rapidly heating and cooling from about 18° C. to about 38° C.

49. The method of any one of claims 37 to 48, wherein the method comprises updating the target flavor profile based on a region in which the user resides and/or originates.

50. The method of any one of claims 37 to 49, wherein the method comprises updating the target flavor profile based on a scenario presented to the user.

51. The method of any one of claims 37 to 50, wherein the method comprises updating the target flavor profile based on one or more pre-calibrated profiles for the user.

52. The method of any one of claims 37 to 51, wherein the one or more nerves of the user comprises one or more cranial nerves.

53. The method of claim 52, wherein the one or more cranial nerves is or comprises a trigeminal nerve.

54. A system for delivering a simulated flavor sensation to a user, the system comprising one or more sensory modules, wherein the sensory modules comprise:
a smell module for providing a scent corresponding to the simulated flavor sensation to the user and positioned to deliver the scent to the nose of the user; and
a thermal module for providing a thermal stimulus to a portion of the tongue and/or a portion of the upper lip and/or the lower lip of the user and/or a portion of an area between the upper lip and the nose of the user and/or one or more nerves of the user.

55. A system for delivering a simulated flavor sensation to a user, the system comprising one or more sensory modules wherein the sensory modules comprise:
a smell module for providing a scent corresponding to the simulated flavor sensation to the user and positioned to deliver the scent to the nose of the user;
an electric taste module for electrically stimulating the tongue of the user; and
a color projection module for providing visual cues to the user.

56. A method of delivering a simulated flavor sensation to a user, the method comprising:
receiving a target flavor profile corresponding to a simulated flavor sensation; and
stimulating the user based on at least the target flavor profile by:
dispensing, to the nose of the subject, one or more fragrances corresponding to the target flavor profile; and
heating or cooling a portion of the tongue of the user and/or a portion of an upper lip of the user and/or a portion of an area between the upper lip and nose and/or one or more nerves using a thermal module, wherein a temperature of the thermal module corresponds to the target flavor profile.

57. A method of delivering a simulated flavor sensation to a user, the method comprising:
receiving a target flavor profile corresponding to a simulated flavor sensation; and
stimulating the user based on at least the target flavor profile by:
dispensing, to the nose of the subject, one or more fragrances corresponding to the target flavor profile;
electrically stimulating the tongue of the user corresponding to the target flavor profile; and/
projecting one or more colors of light corresponding to the target flavor profile.
